(19) United States
(12) Patent Application Publication
(10) Pub. No.: US 2022/0054561 A1
(43) Pub. Date: Feb. 24, 2022

(54) LACHNOSPIRACEAE MITIGATES AGAINST RADIATION-INDUCED HEMATOPOIETIC/GASTROINTESTINAL INJURY AND DEATH, AND PROMOTES CANCER CONTROL BY RADIATION

(71) Applicants: The University of North Carolina at Chapel Hill, Chapel Hill, NC (US); The Regents of the University of Michigan, Ann Arbor, MI (US)

(72) Inventors: Jenny P.-Y. Ting, Chapel Hill, NC (US); Hao Guo, Hillsborough, NC (US); Liang Chen, Worcester, MA (US); Jason W. Tam, Chapel Hill, NC (US); Vincent B. Young, Ann Arbor, MI (US); Mark Koenigsknecht, Cary, NC (US)

(21) Appl. No.: 17/413,861

(22) PCT Filed: Dec. 16, 2019
(86) PCT No.: PCT/US2019/066606

§ 371 Date: Jun. 14, 2021

Related U.S. Application Data
(60) Provisional application No. 62/779,776, filed on Dec. 14, 2018.

(51) Int. Cl. A61K 35/747; A61K 35/744; A61K 35/741; A61P 39/00; A61P 35/00

(52) U.S. Cl. CPC A61K 35/747; A61K 35/744; A61P 35/00; A61P 39/00; A61K 35/741

(57) ABSTRACT

Disclosed herein are data indicating that specific gut commensal bacteria, and metabolites thereof, can mitigate the outcome of high dose total body irradiation. Based on this, provided herein are methods of mitigating and/or preventing side effects from radiation therapy using short chain fatty acid producing bacterium or metabolites thereof. Cancer and tumor treatments and adjuvant therapies are also provided. Methods of treating and/or mitigating damage to a hematopoietic and/or gastrointestinal system in a subject are also provided using the disclosed adjuvant therapeutic compositions.

### CROSS-REFERENCE TO RELATED APPLICATIONS

This application claims benefit of U.S. Provisional Patent Application Ser. No. 62/779,776, filed Dec. 14, 2018, herein incorporated by reference in its entirety.

### GRANT STATEMENT

This invention was made with government support under Grant No. AI067798 awarded by the National Institutes of Health. The government has certain rights in the invention.

### TECHNICAL FIELD

Disclosed herein are methods and systems for using Lachnospiraceae to mitigate against radiation-induced hematopoietic/gastrointestinal injury and death, and promote cancer control by radiation.

### BACKGROUND

Radiation-induced injury is not only a major side-effect that complicates radiotherapy in approximately 50% of patients with an abdominal or pelvic malignancy, but is also a major threat during accidental exposure or a targeted terror attack. Acute radiation syndrome (ARS) developing from whole-body or significant partial-body irradiation is associated with induction of hematopoietic (HP), gastrointestinal (GI) and cerebrovascular syndrome as well as cutaneous, pulmonary and cardiac toxicity. Damage to the HP component is known to play a major role in mortality, especially in weakening the immune system so that it cannot fend off infections. Another major source of damage stems primarily from GI damage. Collateral damage to GI epithelium can lead to acute radiation enteritis, which is associated with malabsorption, bleeding, pain, diarrhea and malnutrition.

These toxicities prevent optimal cancer treatment and can also lead to chronic complications in patients. The high prevalence of hematopoietic loss and acute radiation enteritis, coupled with the paucity of adequate preventative or therapeutic strategies, underscores the importance of further investigation in this field.

The gastrointestinal tract is inhabited by a large diverse microbial community, which is comprised of 10-100 trillion microorganisms and is collectively referred to as the gut microbiota. In recent years, there has been an explosive growth in the knowledge associating gut microbiome to multiple human diseases, such as inflammatory bowel disease (IBD), type 2 diabetes, intestinal vascular remodeling and neuronal homeostasis. More strikingly, emerging research has shown that cancer immunotherapies, such as anti-CTLA4 and anti-PD-L1 treatment, greatly rely on the gut microbiota. Although the protective role of commensal gut bacteria in human diseases is increasingly being appreciated, there remains a need for further development and understanding with respect to the relationship between microbiota and radiation-induced injury. Moreover, there remains a significant need for improved radiation and cancer therapies.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

Provided herein are methods of mitigating and/or preventing side effects from radiation therapy, including providing a subject to be treated with radiation therapy, and/or a subject already treated with radiation therapy, and administering to the subject a bacterium and/or metabolite thereof, wherein the bacterium comprises one or more bacterial strains capable of producing short chain fatty acids (SCFAs), wherein side effects from radiation therapy are mitigated and/or prevented in the subject.

Likewise, in some embodiments, provided herein are methods of treating a tumor and/or a cancer in a subject, the method comprising administering radiation therapy to a subject in need, and administering to the subject a bacterium and/or metabolite thereof, wherein the bacterium comprises one or more bacterial strains capable of producing SCFAs, wherein the tumor and/or a cancer is treated, wherein the effectiveness of the treatment of the tumor and/or cancer is enhanced as compared to radiation therapy alone.

In some aspects, the bacterium comprises intestinal microbiota. In some aspects, the SCFAs produced by the bacterial strains comprise acetate, butyrate and propionate, optionally wherein the ratio of acetate to butyrate to propionate is about 1:5:50, optionally about 1:5:100. In some embodiments, the bacterium comprises strains selected from Lachnospiraceae, Enterococcus faecalis, Lactobacillus rhamonosusl, and combinations thereof. In some embodiments, the bacterium comprises Lachnospiraceae strains, optionally wherein the Lachnospiraceae strains produce butyrate higher than about 120 μM and propionate higher than about 60 μM. In some aspects, the metabolite comprises one or more tryptophan metabolites.

In some aspects, the subject is suffering from acute radiation syndrome (ARS), hematopoietic (HP) injury, gastrointestinal (GI) injury, cerebrovascular syndrome, cutaneous toxicity, pulmonary toxicity, cardiac toxicity and/or combinations thereof. In some embodiments, administration of the bacterium and/or metabolite thereof effectively attenuates radiation-induced hematopoietic and/or gastrointestinal syndrome. In some aspects, the administration of the bacterium and/or metabolite to the subject occurs before or after radiation therapy. In some aspects, the bacterium and/or metabolite thereof is administered orally or by suppository. In some aspects, the subject is a human, optionally wherein the subject is suffering from a cancer, tumor or related condition.

Also provided are methods of treating and/or mitigating damage to a hematopoietic and/or gastrointestinal system in a subject, the method comprising administering to the subject a bacterium and/or metabolite thereof, wherein the bacterium comprises one or more bacterial strains capable of producing SCFAs. In some embodiments, the administration of the bacterium and/or metabolite to the subject occurs before or after an event causing or potentially causing damage to the hematopoietic and/or gastrointestinal system of the subject. In some aspects, the event causing damage to the hematopoietic and/or gastrointestinal system includes radiation, chemotherapy and/or any event, therapy or exposure causing hematopoietic loss and/or acute radiation enteritis. Administration of the bacterium and/or metabolite thereof can effectively attenuate bone marrow loss due to exposure to radiation, chemotherapy or other therapy.

Correspondingly, also provided herein are adjuvant therapeutic compositions, the compositions comprising a bacterium and/or metabolite thereof, wherein the bacterium comprises one or more bacterial strains capable of producing SCFAs, and a therapeutically acceptable carrier. In some aspects, the bacterium comprises intestinal microbiota. In some aspects, the SCFAs produced by the bacterial strains comprise acetate, butyrate and propionate, optionally wherein the ratio of acetate to butyrate to propionate is about 1:5:50, optionally about 1:5:100. In some embodiments, the bacterium comprises strains selected from Lachnospiraceae, Enterococcus faecalis, Lactobacillus rhamonosusl, and combinations thereof. In some embodiments, the bacterium comprises Lachnospiraceae strains, optionally wherein the Lachnospiraceae strains produce butyrate higher than about 120 μM and propionate higher than about 60 μM. In some aspects, the metabolite comprises one or more tryptophan metabolites. The composition can be configured as an adjuvant to anti-cancer radiation therapy and/or anti-cancer chemotherapy, optionally wherein the composition is configured to treat and/or mitigate damage to a hematopoietic and/or gastrointestinal system in a subject to which it is administered.

Provided herein are also methods of screening bacterial strains for use as an anti-cancer adjuvant therapeutic, the methods comprising providing one or more bacterial strains to be screened, conducting a composite genomic analysis for enzymes required for SCFA synthesis, and identify those bacterial strains with a relatively high gene copy for SCFA producing enzymes. In some aspects, the genes for SCFA producing enzymes comprise mmdA, encoding methylmalonyl-CoA decarboxylase for the succinate pathway; lcdA, encoding lactoyl-CoA dehydratase for the acrylate pathway; pduP, encoding propionaldehyde dehydrogenase for the propanediol pathway; and BCoAT, encoding butyryl-CoA transferase for butyrate biosynthesis. The one or more bacterial strains comprises intestinal microbiota. The SCFA producing enzymes produce SCFAs selected from acetate, butyrate and propionate. The bacterial strains are selected from Lachnospiraceae, Enterococcus faecalis, Lactobacillus rhamonosusl, and combinations thereof.

These and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A through 1D include data showing long-lived TBI survivors harbor a gut microbiota with significantly higher diversity. C57BL/6 mice were treated with or without 9.2 Gy total body irradiation, and survival was monitored for 600 days, as shown in FIG. 1A (Non-TBI control mice, n=6; 9.2 Gy TBI mice, n=20). Fecal samples were collected at day 290 post TBI from TBI survivors or at the same time from age matched Non-TBI controls, with principal coordinate analysis (PCoA) showing microbial unweighted UniFrac compositional differences (FIG. 1B), quantified by UniFrac distance between Non-TBI controls and TBI survivors (FIG. 1C; controls, n=5; survivors, n=5). FIG. 1D is a heatmap showing microbial diversity with abundance of sequenced bacterial operational taxonomic units (OTU). Error bars show SEM, *p<0.05, **p<0.01 determined by log-rank (Mantel Cox) test (FIG. 1A) and Student's t test (FIG. 1C).

FIGS. 2A through 2H include data showing long-lived TBI survivors' gut microbiota reduces TBI-induced death and inflammation. FIG. 2A is an illustration of dirty cage sharing experiment. 6-8 weeks specific pathogen-free (SPF) C57BL/6 mice were kept in the dirty cages from Non-TBI controls or Long-lived TBI survivors. Every week, recipients were changed into fresh dirty cages and the dirty cage sharing process lasted for 8 weeks. Then recipients were treated with total body irradiation. Survival rates (FIG. 2B), clinical scores (FIG. 2C), body weight changes (FIG. 2D) and body temperature changes (FIG. 2E) were monitored for 30 days post TBI. (Non-TBI naïve control mice, n=3; TBI naïve control mice, n=6; TBI Control Recipients, n=20; TBI Survivor Recipients, n=19). Mice were euthanized at day 30 post TBI. Femurs and spleens were collected. Representative images of H&E, cleaved caspase 3 and Ki67 stained femur sections (FIG. 2F) as well as spleen sections (FIG. 2G) are shown. FIG. 2H is a Western blot analysis and densitometry of splenic cleaved caspase 3 protein level from mice described in FIG. 2A (Control Recipients, n=4; Survivor Recipients, n=6). Each lane or symbol represents one mouse. Error bars show SEM, *p<0.05, **p<0.01, *** p<0.001, **** p<0.0001 and n.s. means no significance determined by log-rank (Mantel Cox) test (B) and Student's t test (H).

FIGS. 3A through 3E include data showing dirty cage sharing from survivors induced a diversified microbiome composition and increased Clostridiales. Fecal samples were collected after 8 weeks of dirty cage sharing from Control Recipients and Survivor Recipients as shown in FIG. 2A.

FIG. 3A includes principal coordinate analysis (PCoA) showing microbial unweighted UniFrac compositional differences, quantified by UniFrac distance (FIG. 3B) between Control Recipients and Survivor Recipients (Control Recipients, n=6; Survivor Recipients, n=3). FIG. 3C includes principal coordinate analysis (PCoA) between four groups of dirty cage sharing donors and recipients (Control Donors, n=3; Survivor Donors, n=5; Control Recipients, n=6; Survivor Recipients, n=3). Composite results of substantially changed bacterial groups identified by one-way ANOVA from all sequenced fecal bacteria isolated from donor groups (FIG. 3D) and recipient groups (FIG. 3E). Each lane or symbol represents one mouse. Error bars show SEM, *p<0.05, **p<0.01, *** p<0.001, **** p<0.0001 and n.s. means no significance determined by Student's t test (FIG. 3B) and one-way ANOVA (FIG. 3D and FIG. 3E).

FIGS. 4A through 4I include data showing transferring microbiota from Long-lived TBI survivors protects recipients from TBI-induced death. FIG. 4A includes an illustration of fecal microbiota transplant (FMT) experiment. 6-8 weeks germ-free C57BL/6 mice were treated with a PBS suspension of feces derived from Non-TBI controls or LL-TBI survivors, by oral gavage twice a week for 4 weeks. Then recipients were treated with total body irradiation. Survival rates (FIG. 4B), clinical scores (FIG. 4C), body weight changes (FIG. 4D) and body temperatures (FIG. 4E) were monitored for 30 days post TBI (Control Recipients, n=11; Survivor Recipients, n=12). Fecal samples were collected after 4 weeks of FMT from Control Recipients and Survivor Recipients. FIG. 4F includes principal coordinate analysis (PCoA) showing microbial unweighted UniFrac compositional differences, quantified by UniFrac distance (FIG. 4G) between recipient groups (Control Recipients, n=6; Survivor Recipients, n=6). FIG. 4H shows the results of linear discriminative analysis (LDA) effect size (LEfSe) analysis of taxonomic biomarkers identified within Control Recipients and Survivor Recipients. The first eight bars extending right are indicative of enrichment within Survivor Recipients, whereas bottom five bars extending left are indicative of enrichment within Control Recipients. Only taxa meeting an LDA significant threshold (log 2)>±0.2 are show. FIG. 4I shows volcano plots of the relative abundance distribution of microbial OTUs. The x axe shows log twofold of relative abundance ratio between Survivor Recipients and Control Recipients. The y axe shows microbial OTU percentage. Error bars show SEM, *p<0.05, **p<0.01, *** p<0.001, **** p<0.0001 and n.s. means no significance determined by log-rank (Mantel Cox) test (FIG. 4B) and Student's t test (FIG. 4G).

FIGS. 5A through 5I include data showing administration of Lachnospiraceae attenuates radiation-induced inflammation and death. FIG. 5A is a schematic of Lachnospiraceae (Lachno) vs. control (BHI) treatment of 6-8 weeks SPF C57BL/6 mice. After 9 weeks of Lachno/BHI treatment, recipients received total body irradiation. Survival rates (FIG. 5B), clinical scores (FIG. 5C), body weight changes (FIG. 5D) and body temperatures (FIG. 5E) were monitored for 30 days post TBI (BHI Recipients, n=6; Lachno Recipients, n=7). Mice were euthanized at day 1 or day 30 post TBI. Femurs, spleens (FIG. 5F), colons as well as small intestines (FIG. 5G) were collected. Representative images of H&E stained sections are shown. FIG. 5H shows Western blot analysis and densitometry of intestinal proteins were assessed from mice at day 30 post TBI (BHI Recipients, n=4; Lachno Recipients, n=5). Each lane or symbol represents one mouse. FIG. 5I shows the results of gut permeability assay. At day 1 and day 30 post TBI, mice were fasted without water supplement for 4 h followed by orally gavaged with fluorescein isothiocyanate conjugated 4 kDa dextran (FITC-dextran). 2 h later, fluorescence in serum was measured (Non-TBI controls, n=4; BHI Recipients, n=3; Lachno Recipients, n=4; excitation, 490 nm; emission, 520 nm). Error bars show SEM, *p<0.05, **p<0.01, *** p<0.001, **** p<0.0001 and n.s. means no significance determined by log-rank (Mantel Cox) test (FIG. 5B) and Student's t test (FIGS. 5G, 5I).

FIGS. 6A and 6B present data showing SCFAs concentrations in the culture medium of Lachnospiraceae strains. Individual Lachnospiraceae strains were grown anaerobically for 7 days. Culture supernatants were then collected and ¹³C₁-butyrate (Sigma-Aldrich, St. Louis, Mo.) was added to serve as an internal standard for the extraction efficiency of butyrate. Proteins were removed from the supernatant by centrifugation through a 3-kDa spin-filter. Flow through was then analyzed for butyrate, isobutyrate, propionate and lactate content by HPLC separation with subsequent detection by an Agilent 6520 AccurateMass Q-TOF mass spectrometer operating in negative mode (Santa Clara, Calif.). Peak areas were calculated using MassHunter Workstation software. Chromatographic peaks were integrated for samples and areas were compared to peak area for standards (100 μM) for each compound. Lachnospiraceae strains 8, 9, and 21 are low SCFAs producers and strains 2, 14, and 20 are high SCFAs producers, the results of which hare shown in FIG. 6A. In FIG. 6B, 6-8 weeks specific pathogen-free (SPF) C57BL/6J mice first received antibiotics treatment (20 mg/mouse streptomycin) by oral gavage. One day later, mice were orally gavaged with different Lachnospiraceae stains (high or low SCFAs producers). 7 days later, recipients were treated with 2% dextran sulfate sodium (DSS.) Body weight change were monitored, the results of which are shown in FIG. 6B. Error bars show SEM, *p<0.05 determined by two-way ANOVA analysis.

FIGS. 7A through 7H include data showing that Butyrate does not fully replicate the effect of Lachnospiraceae in ameliorating acute radiation syndrome. Butyrate production was determined by Mass Spectrometry from Non-TBI controls versus LL-TBI survivors (FIG. 7A), Control Recipients versus Survivor Recipients from dirty cage sharing expt. as shown in FIG. 2A (FIG. 7B), Control Recipients versus Survivor Recipients from FMT in GF mice expt. as shown in FIG. 4A (FIG. 7C). FIG. 7D includes a schematic of butyrate treatment of 6-8 weeks SPF C57BL/6 mice. After 8 weeks of butyrate treatment, recipients received total body irradiation. Survival rates (FIG. 7E), clinical scores (FIG. 7F), body weight changes (FIG. 7G) and body temperatures (FIG. 7H) were monitored for 30 days post TBI. (Control Recipients, n=14; Butyrate Recipients, n=16).

FIGS. 8A through 8F include data showing that Lachnospiraceae improves therapeutic efficacy of irradiation in tumor models. FIG. 8A is a schematic of short-term Lachnospiraceae/BHI treatment combined with radiotherapy in melanoma tumor models. B16 cells were subcutaneously injected into 6-8 weeks SPF C57BL/6 mice. Four days later, tumor-bearing mice were treated with antibiotics followed by Lachnospiraceae or BHI treatment for three times. Then, 10 Gy X Ray irradiation was operated to tumors locally. Survival rates (FIG. 8B), and tumor volumes (FIG. 8C) were monitored for 25 days post tumor inoculation. Mice were euthanized if tumor reaches 300 mm² and tumor volume was kept in plot as the same volume at endpoint. FIG. 8D is a schematic of long-term Lachnospiraceae/BHI treatment combined with radiotherapy in melanoma tumor models. 6-8 weeks SPF C57BL/6 mice were treated with Lachnospiraceae strains or BHI by oral gavage twice a week for 9 weeks. B16 cells were then subcutaneously injected and mice were monitored for 10 days until most of the tumors grew around 10 mm×10 mm. Then, 10 Gy X Ray irradiation was operated to tumors locally. Survival rates (FIG. 8E), and tumor volumes (FIG. 8F) were monitored for 30 days post tumor inoculation. Mice were euthanized if tumor reaches 300 mm² and tumor volume was kept in plot as the same volume at endpoint. Error bars show SEM, p (n.s.) determined by log-rank (Mantel Cox) test (E) and Mann Whitney test (FIG. 8F).

FIG. 9 depicts data for relative genomic DNA copy number of the key enzymes of propionate and butyrate synthesis normalized to total bacterial 16S rRNA gene copy number in the feces from mice treated with Lachno or BHI (WT Lachno, n=9; Nlrp12^{-/-} Lachno, n=9; WT BHI, n=19; Nlrp12⁻⁻ BHI, n=17).

FIGS. 10A through 10C include data showing that the radioprotective function of Lachnospiraceae dependents on SCFAs production ability. FIG. 10A is a schematic of Lachno-high SCFA producer versus Lachno-low SCFA producer transfer experiment. Six-eight weeks specific pathogen-free (SPF) C57BL/6J mice first received antibiotic treatment (20 mg/mouse streptomycin) by oral gavage. One day later, mice were orally gavaged with either high producer strains or low producer strains twice a week for 8 weeks. 8.2 Gy lethal dose TBI were performed to all recipients. FIGS. 10B and 10C show survival rate and clinical scores were monitored for 30 days post TBI. Error bars show SEM, *p<0.05, ****p<0.0001 determined by log-rank (Mantel Cox) test (FIG. 10B), and Mann-Whitney test for area under the curve (AUC) (FIG. 10C). Data were combined from two independent experiments.

FIGS. 11A through 11F include data showing that commensal-associated short chain fatty acids suppress radiation-induced death and damage. FIG. 11A is a schematic of short chain fatty acids (SCFAs) treatment. Survival rates (FIG. 11B) and clinical scores (FIG. 11C) were monitored for 30 days. Femurs and spleens were stained for H&E and quantified for BM cellularity and spleen EMH scores (FIG. 11D). White pulp (WP, black dash circles), red pulp (RP, area between black solid lines), and megakaryocytes (black arrows) are shown. FIG. 11 E shows flow cytometric analysis of hematopoietic stem and progenitor cells (HSPC, gated as Lin⁻Sca1⁺c-kit⁺), common myeloid progenitors (CMP, gated as Lin⁻Sca1⁻ckit⁺CD16/32^{int}), granulocyte-macrophage progenitors (GMP, gated as Lin⁻Sca1⁻ckit⁺CD16/32^{hi}) and megakaryocyte-erythroid progenitors (MEP, gated as Lin⁻Sca1⁻ckit⁺CD16/32^{lo}) from BM. Total CMP, GMP and MEP percentages of Lin⁻ cells are shown in the right histogram. Colon samples were stained with AB/PAS for mucus layer and goblet cells, as shown in FIG. 11F. Representative images are shown. Mucus layer is indicated by area between dash lines and crypt length is indicated by double-headed arrow. Mucus layer thickness and crypt length were quantified. Error bars show SEM, *p<0.05, **p<0.01, *** p<0.001 determined by log-rank (Mantel Cox) test (B), Mann-Whitney test for area under the curve (AUC) (C) and Student's t test (D, E, F).

FIGS. 12A through 12C include data showing that special combinations of short chain fatty acids have better protection against radiation-induced syndrome. FIG. 12A is a schematic of short chain fatty acids (SCFAs) combination treatment. Survival rates (FIG. 12B) and clinical scores (FIG. 12C) were monitored for 30 days. Error bars show SEM, *p<0.05, **p<0.01, *** p<0.001 determined by log-rank (Mantel Cox) test (FIG. 12B) and Mann-Whitney test for area under the curve (AUC) (FIG. 12C).

FIGS. 13A through 13C include data showing that Enterococcus faecalis and Lactobacillus rhamonosus protect SPF recipients from TBI-induced death. FIG. 13A is a schematic of Enterococcus faecalis, Bacteroides fragilis, Lactobacillus rhamonosus versus control (BHI medium) transfer experiment. Six-eight weeks specific pathogen-free (SPF) C57BL/6J mice first received antibiotic treatment (20 mg/mouse streptomycin) by oral gavage. One day later, mice were orally gavaged with indicated bacteria strains separately twice a week for 8 weeks. BHI medium was used as a vehicle control. 8.2 Gy lethal dose TBI were performed to all recipients. FIGS. 13B and 13C show where survival rate and clinical scores were monitored for 30 days post TBI. Error bars show SEM, *p<0.05 determined by log-rank (Mantel Cox) test (FIG. 13B), and Mann-Whitney test for area under the curve (AUC) (FIG. 13C). Data were combined from two independent experiments.

FIGS. 14A through 14G include data showing that untargeted metabolomics reveals tryptophan metabolites as potent radio-protectants. Metabolite profiles were measured in fecal samples of AM-Ctrl and ES mice at Day 290 post TBI. Total ion chromatogram (TIC) metabolomic cloudplot (p<0.01) (FIG. 14A) and PCA score plot (14B) show distinct metabolites separation between these two groups. FIG. 14 C shows metabolite set enrichment analysis (MSEA) was conducted to identify and interpret patterns of metabolites in biochemical contexts. In FIG. 14D, metabolic network graphs (MetaMapp) were generated to integrate the biochemical pathways and chemical relationships of all detected metabolites. Identified metabolites are represented by circle nodes, with lower transparency indicating lower p-values from Welch's t-test. Lighter grey nodes denote metabolites with higher abundance in ES group; darker grey nodes denote those higher in AM-Ctrl group. Solid grey lines connecting distinct metabolites symbolize KEGG reactant pair links; dashed grey lines symbolize chemical similarity with a Tanimoto coefficient score >0.7. Tryptophan metabolites are highlighted by a large shadow (labelled), while other metabolite families are distinguished by separate shadowed areas. FIG. 14E is a schematic of tryptophan metabolites treatment. Survival rates (FIG. 14F) and clinical scores (FIG. 14G) were monitored for 30 days. Error bars show SEM, *p<0.05, **p<0.01, ***p<0.001 determined by log-rank (Mantel Cox) test (FIG. 14F) and Mann-Whitney test for area under the curve (AUC) (FIG. 14G).

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. DEFINITIONS

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a unit cell” includes a plurality of such unit cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

### III. DISCUSSION

Summarily, the data herein show that after exposure to lethal dose total body irradiation (TBI), about 5-20% of C57BL/6J mice successfully recovered from radiation-induced damage. By using high-throughput gene-sequencing analysis of 16S rRNA, the microbiota composition in both the survivors and controls was identified. As shown herein, it was discovered that survivors harbored a gut microbiota with significantly higher diversity and distinct community composition relative to that in controls. Then two different fecal microbiota exchange experiments were conducted (i) by housing recipients in the dirty cages, which previously housed long-lived TBI survivors or age-matched non-TBI controls (donors) and contained fecal materials from these two donor groups; (ii) by transferring fecal microbiota from long-lived TBI survivors or age-matched non-TBI controls (donors) to recipients via oral gavage. Upon total body irradiation, recipients who received survivors' microbiota showed dramatically higher protection against TBI-induced injury and death. 16S rRNA sequencing analysis identified a significant decrease in abundance of Erysipelotrichaceae family as well as increases in the abundance of Bacteroidales and Clostridiales orders in survivor recipients compared with that in control recipients. Among these families, Lachnospiraceae was selected as a more abundant bacterium in the survivors group. To further examine the possibility of using Lachnospiraceae as a countermeasure against radiation-induced damage, these bacteria were cultured in vitro and reconstituted to SPF mice by oral gavage. Lachnospiraceae efficiently increased mice survival and decreased HP as well as GI syndromes in recipients post TBI. Furthermore, the function of butyrate, which is a commonly studied metabolite that is also produced by Lachnospiraceae, was detected and we found that this short chain fatty acid had radiomitigation properties albeit less than Lachnospiraceae strains. Moreover, we also found that Lachnospiraceae modestly improved the efficacy of localized radiotherapy by slowing down tumor growth as well as improving mice survival in a melanoma model. Taken together, we elucidated the role of the intestinal microbiota as an integrative point in the pathogenesis of acute radiation syndrome, and found a specific bacterium, Lachnospiraceae, that protects against radiation injury.

Currently, only one promising radiation countermeasure has been approved by the U.S. FDA as an effective countermeasure for ARS. In 2015, G-CSF was approved as a drug by the FDA for treating radiation-induced hematopoietic damage. It has also been approved by the Centers for Disease Control and Prevention for administration to victims exposed to a radiological nuclear incident. However, G-CSF has been shown to increase the survival of irradiated mice only when injected subcutaneously daily from day 1 to 16 (16 doses). The recommended dosage of commercial G-CSF (Filgrastim, Neupogen) in cancer patients undergoing bone marrow transplantation is 10 mcg/kg/day given as an intravenous infusion no longer than 24 hours and continue for several days until absolute neutrophil count increases beyond 10,000/mm³, which makes it quite costly, inconvenient to use and limits its clinical application. Furthermore, side effects are also a big concern. G-CSF administration may cause fever, myalgia, respiratory distress, hypoxia, splenomegaly, sickle cell crisis and incidences of Sweet's syndrome (acute febrile neutropenia dermatosis/skin plaques). Moreover, there are several lines of evidence showing that cancer patients who received G-CSF treatment had an increased risk of developing myelodysplasia (MDS) and acute myeloid leukemia (AML). On the other hand, Lachnospiraceae can be cultured in anaerobe culturing devices at a large scale, making it readily available and inexpensive. By using standard lyophilization method and encapsulation into enteric capsules, it is stable for easy handling, transporting, storage as well as oral administration with rapid reconstitution in the intestine. Here we show that Lachnospiraceae resulted in increased hematopoietic recovery and gastrointestinal wound repair. In addition, it is shown herein that the bacteria did not accelerate tumor growth, thus eliminating the possibility of this unintended consequence of using this bacteria strain to treat either accidental exposure to radiation or intentional exposure during radiation therapy for cancer. In contrast, the data herein unexpectedly showed that Lachnospiraceae and radiation provide better control of tumor growth, thus the bacteria may be used in conjunction with radiation to control cancer. Considering all these features, Lachnospiraceae and its metabolites represent appealing and cost-effective alternatives to conventional G-CSF or other radio-countermeasures for ARS caused by either radiotherapy or deliberate/accidental radiation release. Equally important, it might improve the outcome of radiation therapy to control cancer.

Thus, in some embodiments, provided herein are methods of mitigating and/or preventing side effects from radiation therapy. Such methods can comprise providing a subject to be treated with radiation therapy, and/or a subject already treated with radiation therapy, and administering to the subject a bacterium and/or metabolite thereof, wherein the bacterium comprises one or more bacterial strains capable of producing short chain fatty acids (SCFAs), wherein side effects from radiation therapy are mitigated and/or prevented in the subject. In some embodiments, the bacterium comprises intestinal microbiota. In some embodiments, the bacterium comprises Lachnospiraceae strains, optionally wherein the Lachnospiraceae strains produce butyrate higher than about 120 μM and propionate higher than about 60 μM. In some embodiments, the subject is suffering from acute radiation syndrome (ARS), hematopoietic (HP) injury, gastrointestinal (GI) injury, cerebrovascular syndrome, cutaneous toxicity, pulmonary toxicity, cardiac toxicity and/or combinations thereof.

In some embodiments, administration of the bacterium and/or metabolite thereof effectively attenuates radiation-induced hematopoietic and/or gastrointestinal syndrome. In some embodiments, the administration of the bacterium and/or metabolite to the subject occurs before or after radiation therapy. In some embodiments, the bacterium and/or metabolite thereof is administered orally or by suppository. In some embodiments, the subject is a human, optionally wherein the subject is suffering from a cancer, tumor or related condition.

Also provided herein are methods of treating a tumor and/or a cancer in a subject, comprising administering radiation therapy to a subject in need, and administering to the subject a bacterium and/or metabolite thereof, wherein the bacterium comprises one or more bacterial strains capable of producing SCFAs, wherein the tumor and/or a cancer is treated, wherein the effectiveness of the treatment of the tumor and/or cancer is enhanced as compared to radiation therapy alone. In some embodiments, the bacterium comprises intestinal microbiota. In some embodiments, the bacterium comprises Lachnospiraceae strains, optionally wherein the Lachnospiraceae strains produce butyrate higher than about 120 μM and propionate higher than about 60 μM. In some embodiments, administration of the bacterium and/or metabolite thereof effectively attenuates radiation-induced hematopoietic and/or gastrointestinal syndrome. In some embodiments, the administration of the bacterium and/or metabolite to the subject occurs before or after radiation therapy. In some embodiments, the bacterium and/or metabolite thereof is administered orally or by suppository. In some embodiments, the subject is a human, optionally wherein the subject is suffering from a cancer, tumor or related condition.

Still yet, in some aspects, provided herein are methods of treating and/or mitigating damage to a hematopoietic and/or gastrointestinal system in a subject, the method comprising administering to the subject a bacterium and/or metabolite thereof, wherein the bacterium comprises one or more bacterial strains capable of producing SCFAs. In some embodiments, the administration of the bacterium and/or metabolite to the subject occurs before or after an event causing or potentially causing damage to the hematopoietic and/or gastrointestinal system of the subject. In some embodiments, the event causing damage to the hematopoietic and/or gastrointestinal system includes radiation, chemotherapy and/or any event, therapy or exposure causing hematopoietic loss and/or acute radiation enteritis. In some embodiments, administration of the bacterium and/or metabolite thereof effectively attenuates bone marrow loss due to exposure to radiation, chemotherapy or other therapy. In some embodiments, the bacterium comprises intestinal microbiota. In some embodiments, the bacterium comprises Lachnospiraceae strains, optionally wherein the Lachnospiraceae strains produce butyrate higher than about 120 μM and propionate higher than about 60 μM.

Also provided herein are adjuvant therapeutic compositions, comprising a bacterium and/or metabolite thereof, wherein the bacterium comprises one or more bacterial strains capable of producing SCFAs, and a therapeutically acceptable carrier. In some embodiments, the bacterium comprises intestinal microbiota. In some embodiments, the bacterium comprises Lachnospiraceae strains, optionally wherein the Lachnospiraceae strains produce butyrate higher than about 120 μM and propionate higher than about 60 μM. In some embodiments, the composition is configured as an adjuvant to anti-cancer radiation therapy and/or anti-cancer chemotherapy, optionally wherein the composition is configured to treat and/or mitigate damage to a hematopoietic and/or gastrointestinal system in a subject to which it is administered.

Methods of screening bacterial strains for use as an anti-cancer adjuvant therapeutic are also provided herein. Such methods comprise providing one or more bacterial strains to be screened, conducting a composite genomic analysis for enzymes required for SCFA synthesis, and identify those bacterial strains with a relatively high gene copy for SCFA producing enzymes, e.g. at least 10%, 15%, 20%, 25%, 30%, 35%, 40%, 45%, 50%, 75% or 90% increased gene copy for SCFA producing enzymes as compared to other bacterial strains. In some embodiments, the genes for SCFA producing enzymes comprise mmdA, encoding methylmalonyl-CoA decarboxylase for the succinate pathway; lcdA, encoding lactoyl-CoA dehydratase for the acrylate pathway; pduP, encoding propionaldehyde dehydrogenase for the propanediol pathway; and BCoAT, encoding butyryl-CoA transferase for butyrate biosynthesis.

a. Pharmaceutical/Adjuvant Therapeutic Compositions

The compounds disclosed herein can be formulated in accordance with the routine procedures adapted for a desired administration route. Accordingly, in some embodiments, the presently disclosed subject matter provides an adjuvant therapeutic composition, or pharmaceutical composition, comprising a therapeutically effective amount of a compound as disclosed hereinabove (e.g., a bacterium and/or metabolite thereof, wherein the bacterium comprises one or more bacterial strains capable of producing SCFAs). The therapeutically effective amount can be determined by testing the compounds in an in vitro or in vivo model and then extrapolating therefrom for dosages in subjects of interest, e.g., humans. The therapeutically effective amount should be enough to exert a therapeutically useful effect in the absence of undesirable side effects in the subject to be treated with the composition.

Pharmaceutically acceptable carriers are well known to those skilled in the art and include, but are not limited to, from about 0.01 to about 0.1M and preferably 0.05M phosphate buffer or 0.8% saline. Such pharmaceutically acceptable carriers can be aqueous or non-aqueous solutions, suspensions and emulsions. Examples of non-aqueous solvents suitable for use in the presently disclosed subject matter include, but are not limited to, propylene glycol, polyethylene glycol, vegetable oils such as olive oil, and injectable organic esters such as ethyl oleate. Aqueous carriers suitable for use in the presently disclosed subject matter include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the presently disclosed subject matter can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Parenteral carriers suitable for use in the presently disclosed subject matter include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the presently disclosed subject matter can be mixed as needed with disintegrants, diluents, granulating agents, lubricants, binders and the like using conventional techniques known in the art. The carriers can also be sterilized using methods that do not deleteriously react with the compounds, as is generally known in the art. The compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Further, formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder.

The compounds can be formulated as a suppository, with traditional binders and carriers such as triglycerides.

Oral formulations can include standard carriers such as pharmaceutical grades of mannitol, lactose, starch, magnesium stearate, polyvinyl pyrollidone, sodium saccharine, cellulose, magnesium carbonate, etc.

In some embodiments, the pharmaceutical composition comprising the compound of the presently disclosed subject matter can include an agent which controls release of the compound, thereby providing a timed or sustained release compound.

b. Methods of Treatment

As described hereinabove, provided herein are methods of mitigating and/or preventing side effects from radiation therapy, and/or methods of treating a tumor and/or a cancer in a subject, comprising administering radiation therapy to a subject in need, and administering to the subject a bacterium and/or metabolite thereof. Also provided are methods of treating and/or mitigating damage to a hematopoietic and/or gastrointestinal system in a subject.

An effective amount of the compounds disclosed herein, e.g., a bacterium and/or metabolite thereof, wherein the bacterium comprises one or more bacterial strains capable of producing SCFAs, comprise amounts sufficient to produce a noticeable effect, such as, but not limited to, substantially preventing and/or mitigation hematopoietic loss and/or acute radiation enteritis caused by radiation, chemotherapy and/or any event, therapy or exposure causing such deleterious effects. In some embodiments, an effective amount of the compounds disclosed herein, e.g., a bacterium and/or metabolite thereof, comprises amounts sufficient to produce a noticeable effect, such as, but not limited to, substantially attenuating bone marrow loss due to exposure to radiation, chemotherapy or other therapy.

Actual dosage levels of active ingredients in a therapeutic compound of the presently disclosed subject matter can be varied so as to administer an amount of the active compound that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

The therapeutically effective amount of a compound can depend on a number of factors. For example, the species, age, and weight of the subject, the precise condition requiring treatment and its severity, the nature of the formulation, and the route of administration are all factors that can be considered.

A compound of the presently disclosed subject matter can also be useful as adjunctive, add-on or supplementary therapy for the treatment of the above-mentioned diseases/disorders, e.g. an adjuvant to radiation and/or chemotherapy for treating a cancer or tumor. Said adjunctive, add-on or supplementary therapy means the concomitant or sequential administration of a compound of the presently disclosed subject matter to a subject who has already received administration of, who is receiving administration of, or who will receive administration of one or more additional therapeutic agents for the treatment of the indicated conditions, for example, radiation and/or chemotherapy.

c. Subjects

The subjects treated, tested or from which a sample is taken, is desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which screening is desirable, particularly agricultural and domestic mammalian species.

The disclosed methods are particularly useful in the treating, testing and/or screening of warm-blooded vertebrates. Thus, the presently disclosed subject matter concerns mammals and birds.

More particularly, provided herein is the testing, screening and/or treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, provided herein is the treatment of livestock, including, but not limited to, domesticated swine (pigs and hogs), ruminants, horses, poultry, and the like.

In some embodiments, the subject to be used in accordance with the presently disclosed subject matter is a subject in need of treatment and/or diagnosis. In some embodiments, a subject can be in need of, or currently receiving, a radiation therapy.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Intestinal Microbiota Potently Protect Against Total Body Irradiation-Induced Lethal Injury and Death

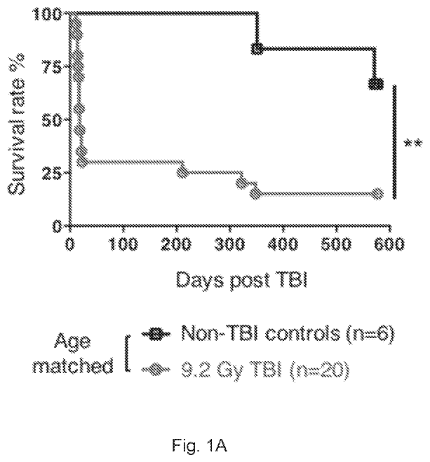

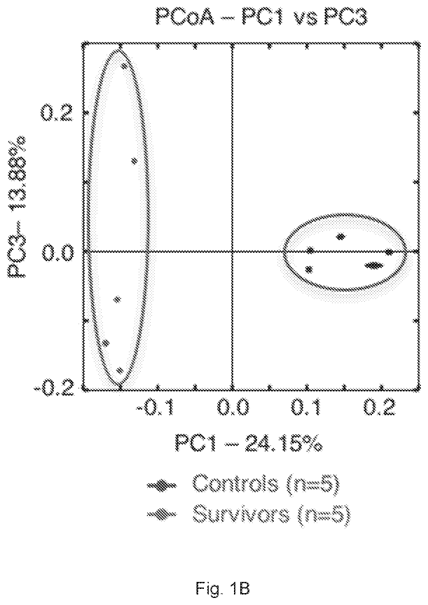

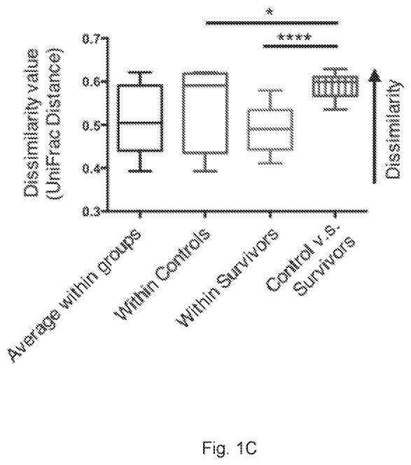

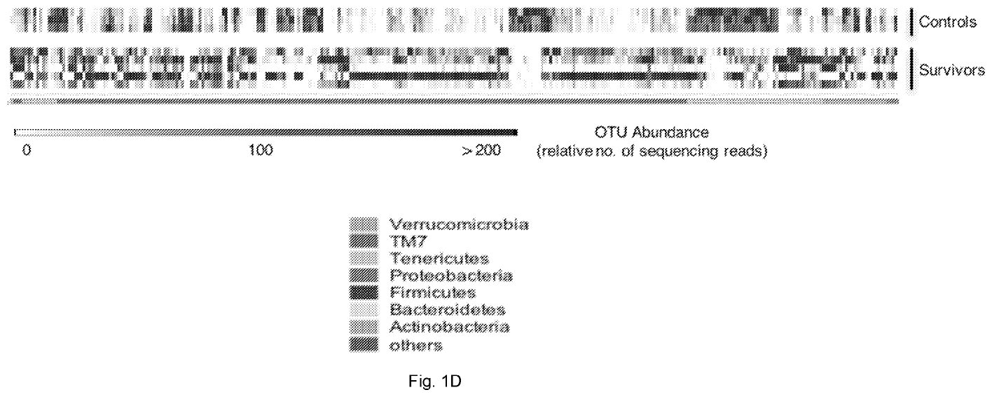

C57BL/6 mice are highly sensitive to a lethal dose of total body irradiation²⁶, however approximately 5-20% of mice survived and recovered within 30 days and lived for more than 600 days (FIG. 1A). Strikingly, magnetic resonance Imaging (MRI) analysis showed these long-lived survivors had no tumors or physiologic changes in brain, gut, kidney or spleen²⁶. As such, to determine if the gut microbiome is different in these survivors, high-throughput gene-sequencing analysis of 16S rRNA gene expression in fecal bacterial DNA isolated from age-matched non-TBI control mice (controls) and long-lived 9.2 Gy TBI super survivors (survivors) was performed after irradiation exposure on day 290, the results of which are shown herein. Rarefaction analysis was assessed to compare bacterial diversity within individual mice of these two groups. As shown in FIGS. 1B and C, survivors harbored a gut microbiota with significantly higher diversity and distinct community composition relative to that of controls. Comparison of within- and between-groups dissimilarity indicated that the microbiome difference between controls and survivors was significantly greater than the difference within each group (FIG. 1C, calculated from FIG. 1B). This result was further supported by a heatmap of bacterial operational taxonomic units (OTUs) where more groups of bacteria were found in survivors than in controls (FIG. 1D). These results prompted further investigation based on the discovery herein that changes in the intestinal bacterial communities were able to influence radio-sensitivity in C57BL/6 mice.

### Example 2

### Fecal Microbiota Exchange Protects Against Radiation-Induced Death and Hematopoietic Toxicity

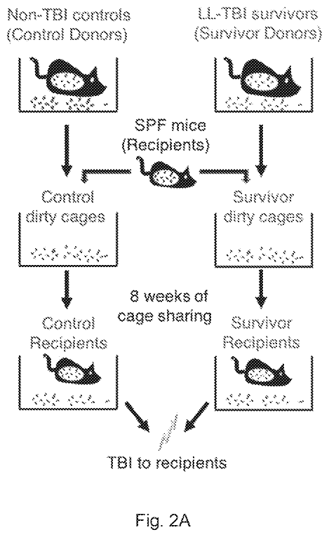

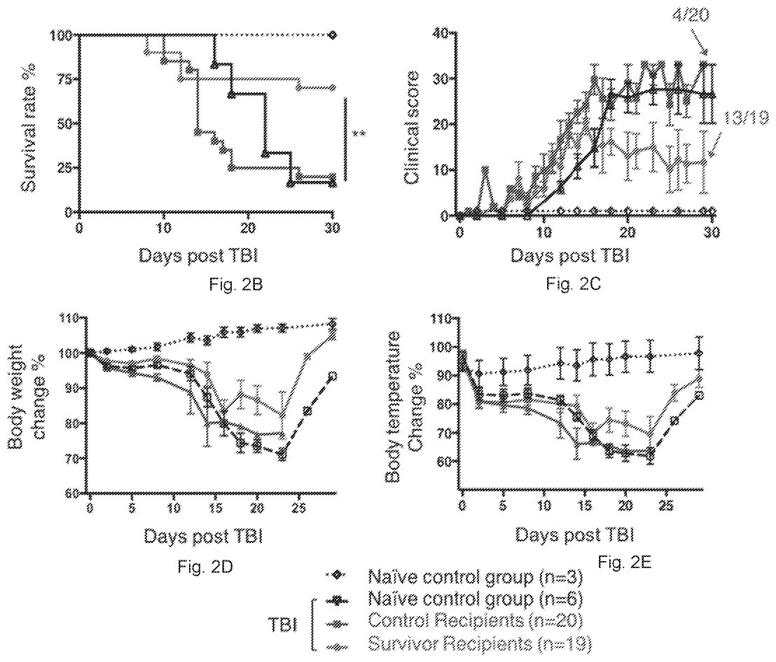

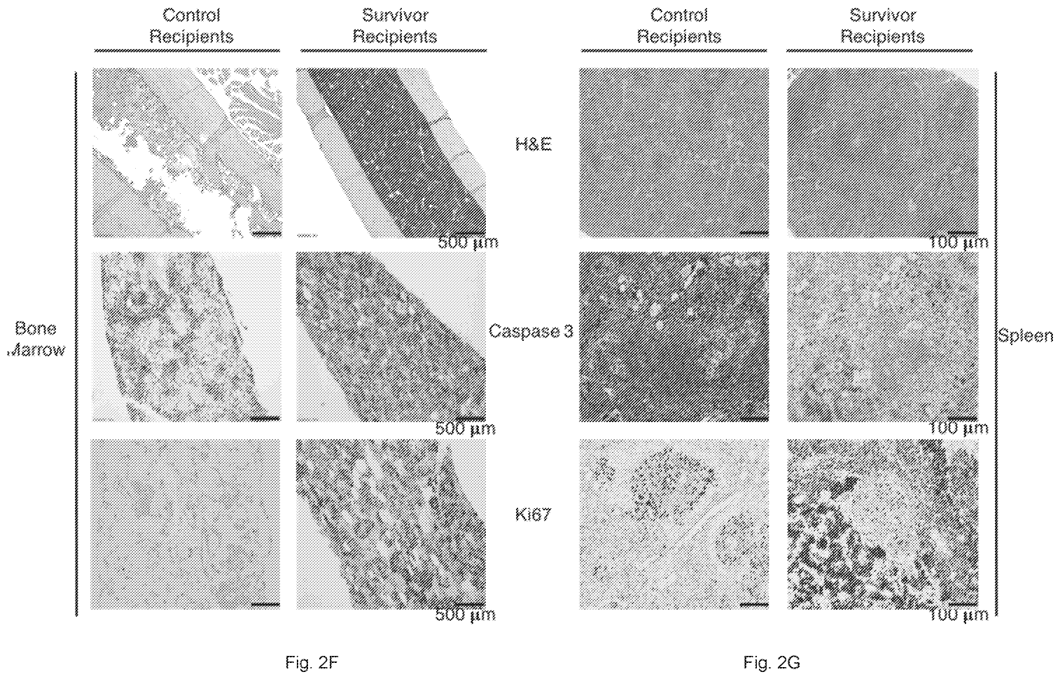

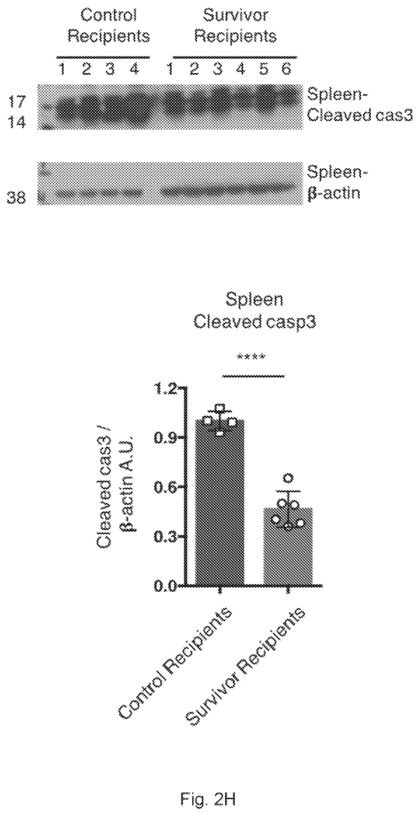

Divergent factors, such as housing, diet and inflammation states, can dramatically affect enteric microbiota^{17,27,28}. Therefore, to more stringently investigate the contribution of gut microbiota in radio-protection, a strategy was designed where cages which housed the super survivors were subsequently used to house mice which were scheduled for radiation later (FIG. 2A). As the initial experiments were performed with male mice to avoid the impact of the estrus cycle, the traditional cohousing experiment was not appropriate to render microbiota exchange between donors and recipients since the combination of aging super survivors with young experimental males may cause fighting and possible death to the aging mice. Instead, the dirty cages were reserved, which contained feces and used beddings charged with numerous bacteria, from long-lived TBI survivors as well as age-matched non-TBI controls. Specific pathogen-free (SPF) C57BL/6 mice were used as recipients and kept in those dirty cages. For 8 weeks on a weekly basis, these recipients were changed into fresh dirty cages from long-lived TBI survivors versus age-matched non-TBI controls. The survival of recipients exposed to lethal dose TBI was monitored. Mice started to succumb to radiation effects by approximately 2 weeks post TBI (FIG. 2B), as defined by weight loss >25% and/or a clinical score (encompassing seven body parameters, shown in Table 1.) greater than 15. Most strikingly, 13 of 19 mice (68%) that were recipients of the super survivors microbiome survived for 30 days post TBI compared to only 20% of control recipients (FIG. 2B). The clinical scores and body weight changes as well as temperature changes of survivor recipients were significantly lower than that of control recipients (FIG. 2C-E).

TABLE I
Clinical Score Parameters.

```
Assess the following parameters and tally
with associated scoring system:
A. Physical appearance
  0 - normal
  1 - lack of grooming
  2 - rough hair coat
  3 - very rough hair coat
B. Posture
  0 - normal
  1 - sitting in hunched position
  4 - hunched posture, head resting on floor
  6 - lying prone on cage floor/unable to maintain upright posture
  (**suggests moribund and euthanasia required)
C. Activity/Behavior
  0 -normal
  1 - somewhat reduced/minor changes in behavior
  3 - above plus change in respiratory rate or effort
  6 - moves only when stimulated
D. Appetite
  0 - normal
  1 - reduced appetite
  2 - not eating since last check point
  (**assumes multiple checks per day, by visual
  inspection of food on floor of cage)
  3 - not eating for last 2 check points
  (**assumes multiple checks per day, by visual
  inspection of food on floor of cage)
Measure the parameters:
E. Hydration
  0 - normal
  1 - mildly dehydrated (<1 sec skin tent)
  2 - moderately dehydrated
  (1-2 sec skin tent; **with supplemental fluids
  given by s.c. and hydrogel provided)
  3 - severely dehydrated
  (>2 sec skin tent; **with supplemental fluids
  given by s.c. and hydrogel provided)
F. Body Weight (assessed weekly, then every other day when 10%
weight change reached, and daily after 15% weight change reached)
  0-normal (<5% change from initial weight)
  1 - 5-10% weight change
  2 - 10-14.9% weight change
  3 - 15-19.9% weight change
  4 - 20-24.9% weight change
  6 > 25% weight change
G. Body temperature (ventral surface temp.
determined using infrared thermometer)
  0 - normal (33-35° C.)
  2 - 30-32.9° C
  4 - 28-29.9° C
  6 - < 28° C.
Endpoint for euthanasia with any single parameter of 6 or
combined score for parameters A to G = > 15.
Immediate endpoints for euthanasia:
  1. Unconsciousness
  2. Inability to remain upright
  3. Agonal respiration (i.e. gasping)
  4. Convulsions
```

Total body exposure to 2 Gy or higher radiation induces severe damage in hematopoietic systems including bone marrow and spleen, which might lead to death from infection or hemorrhage within 30 days²⁹. Replenishment of hematopoietic sites is critical for recovery following radiation exposure. In order to gain more insight into the gut microbiota's radio-protection function, histological studies were conducted in bone marrow and spleen samples at day 30 post TBI. Extensive stromal injury and cell death were observed in BM from microbiota recipients of control mice (FIG. 2F). However, femurs from microbiome recipients of super survivors were normal in appearance (95-100% cellularity). Cleaved caspase 3 and Ki67 staining were also conducted in femur samples. Survivor recipients showed dramatically less apoptosis and more proliferation in BM cells as compared with that in control recipients (FIG. 2F). Consistent with BM results, splenic architecture was also substantially normal in survivor recipients, with white pulps containing well-developed lymphocyte-rich follicles and red pulps containing venous sinusoids and scattered hematopoietic elements (FIG. 2G), while appreciable atrophy and lymphocyte depletion were observed in control recipients. Meanwhile, there was also decreased cleaved caspase 3 staining and increased Ki67 staining in spleens of survivor recipients, which was also confirmed by western blot of cleaved caspase 3 protein levels (FIG. 2H). These results indicated hematopoietic system was successfully protected from radiation by microbiota exchange.

### Example 3

### Fecal Microbiota Exchange Results in Diversified Microbiome Composition and Increased Clostridiales

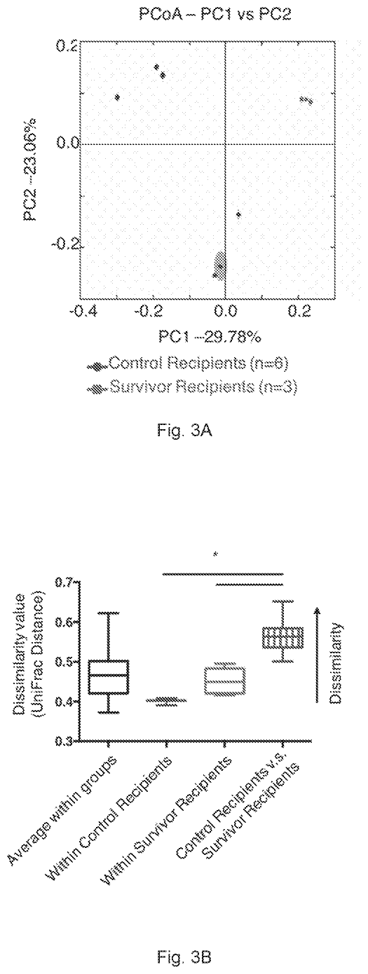

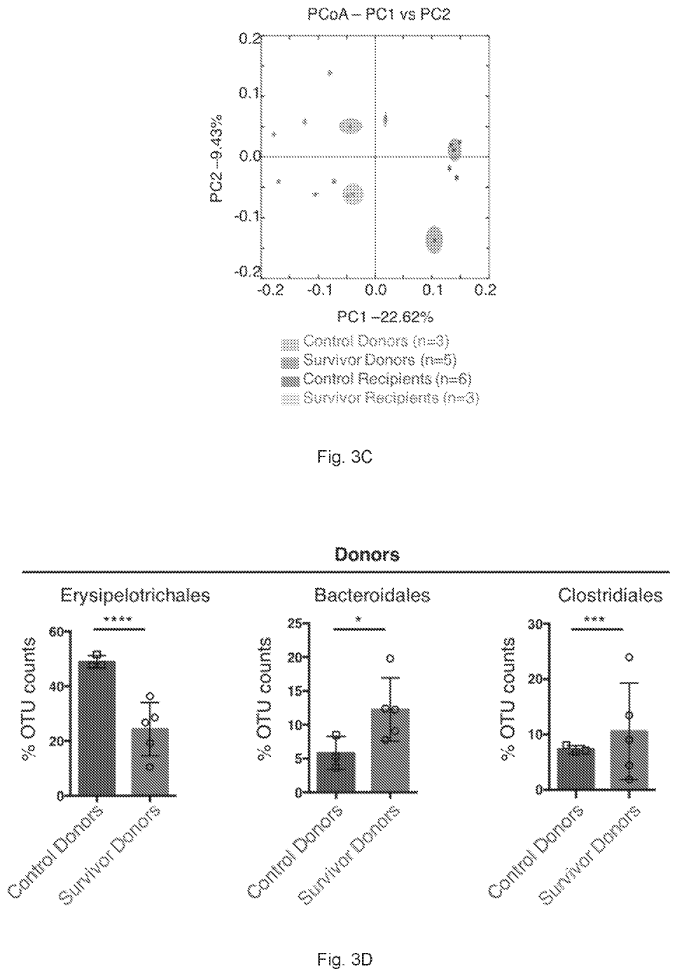

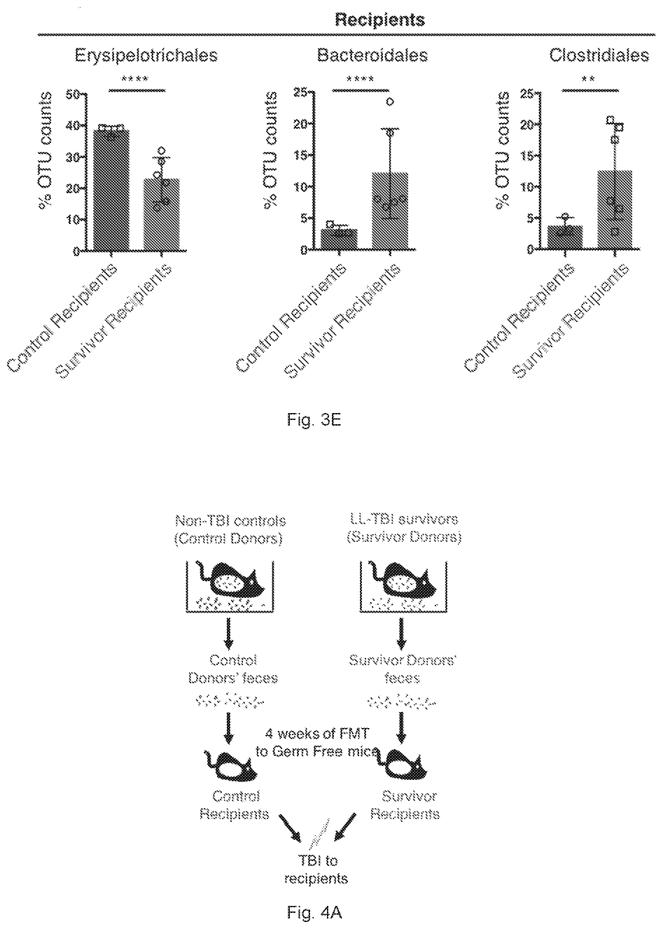

Next, studies were designed to investigate how the gut bacterial composition structure was altered in the dirty cage sharing experiment. To address this question, bacterial 16S rRNA genes were profiled in feces of control recipients and survivor recipients after 8 weeks of dirty cage sharing as shown in FIG. 2A. Dirty cages from long-lived TBI survivors led to a significantly increased microbiome composition when compared between survivor recipients and control recipients, shown by a principal component analysis (PCA) and quantified by UniFrac dissimilarity distance (FIGS. 3A-B). What's more, microbiome compositions in recipient groups were similar to donor groups respectively, suggesting the dirty cage sharing was efficient in exchanging gut microbiota from donors to recipients (FIG. 3C).

To further determine if the transferred microbiota resulted in changes in specific bacteria, one-way analysis of variance (ANOVA) of all results from sequenced fecal bacteria identified by 16S rRNA gene sequencing both in donor and recipient groups was performed. Significant decreases in abundance of the Erysipelotrichaceae family as well as increases in abundance of Bacteroidales and Clostridiales orders were found in long-lived TBI survivors and survivor recipients compared with non-TBI controls and control recipients, respectively (FIGS. 3D-E).

### Example 4

### Fecal Microbiota Transplant Ameliorates Radiation-Induced Death by Altering Gut Bacterial Composition Structure

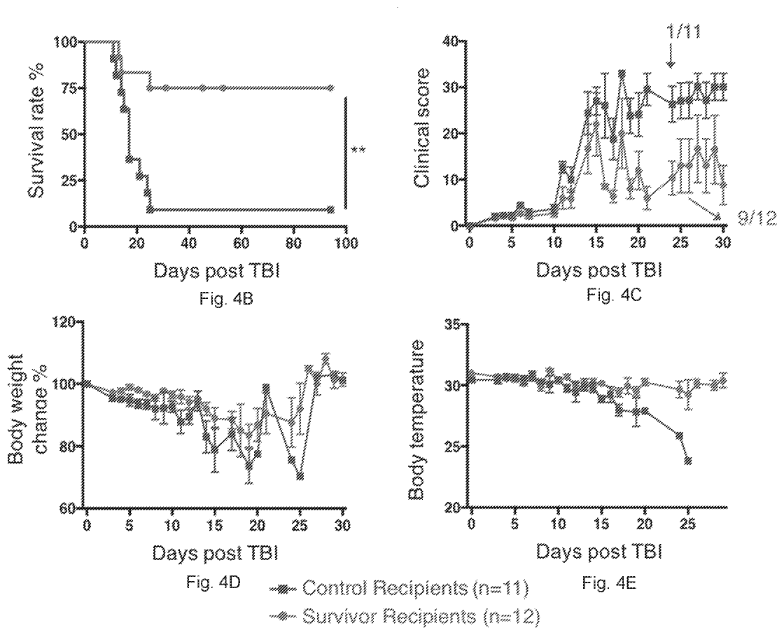

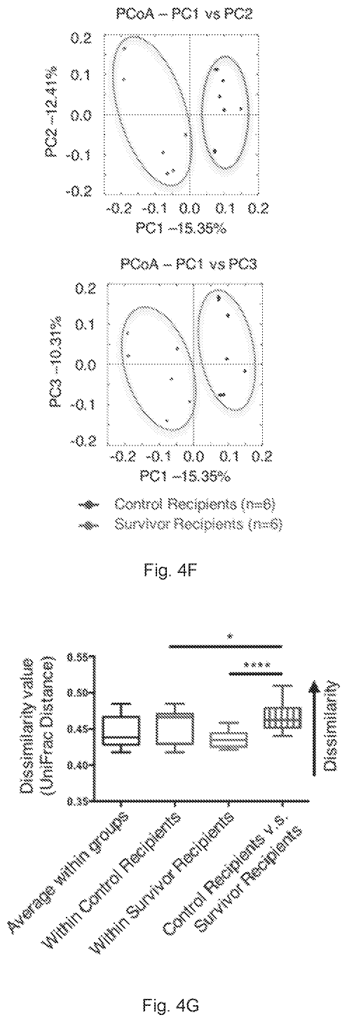

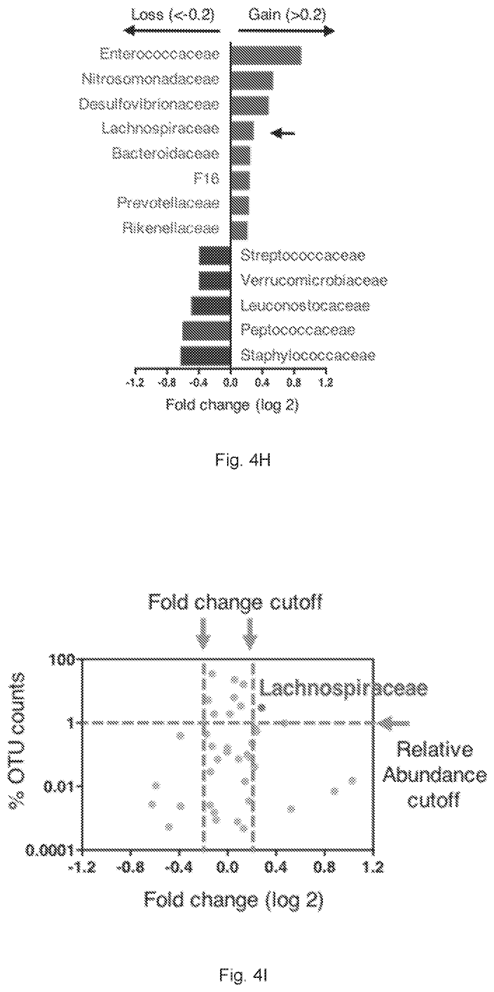

To consolidate the relevance between gut microbiota and radio-sensitivity, a fecal microbiota transplant (FMT) experiment was performed in which germ-free (GF) C57BL/6 mice were reconstituted with the microbiota from long-lived TBI survivors and age-matched non-TBI controls via oral gavage twice a week for 4 weeks, as previously described (FIG. 4A)^{14,18}. Transferring fecal microbiota from survivor donors into GF recipients resulted in significantly elevated survival, lower clinical score, more stable body weight and temperature compared to recipients of age-matched control donors (FIGS. 4B-E).

Consistent with the results obtained in dirty cage sharing experiment (FIG. 3), substantially different composition of microbiota community was observed in survivor recipients relative to that in control recipients (FIG. 4F). The dissimilarity of microbiome between these two recipient groups was distinctly higher than the dissimilarity within each group (FIG. 4G, calculated from FIG. 4F). Individual distinct taxa were then selected for functional studies to assess their potential contribution to radiation-induced syndrome. To this end, direct comparisons between bacteria intensities within survivor recipients and control recipients were conducted. Linear discriminant analysis Effect Size (LEfSe) analysis showed that a total of 13 taxa were enriched in both groups (8 taxa enriched in survivor recipients and 5 in control recipients), with a linear discriminant analysis (LDA) score >0.2 (FIG. 4H). To further define bacterial taxa with high intensity, volcano plot flagged 9 families (10%) of all detected bacteria families (84 in total) with significant changes between survivor recipients and control recipients (fold change (log 2)>±0.2) as long as high OTU abundance. Among these families, Lachnospiraceae was the most represented strain in survivor recipients with OTU>1% together with a linear discrimination analysis (LDA)score (log 2) in survivors/controls that is >0.2 (FIG. 4I).

### Example 5

### Lachnospiraceae Protects Hematopoietic and Gastrointestinal System from Radiation and Shows Beneficial Radiomitigation Properties

As shown in FIGS. 4H-I, Lachnospiraceae was selected as the most likely bacterium which may play a role in mitigating radiation-induced damage and been used as a beneficial radio-countermeasure, based on the following criteria: (i) identifiable to genus or family level with higher intensity in survivors group; (ii) culturable, to be able to study their functions in vitro and in vivo³⁰; (iii) type strains available to ensure reproducibility³⁰; and (iv) previously associated with immune-regulatory effects^{18,30,31}.

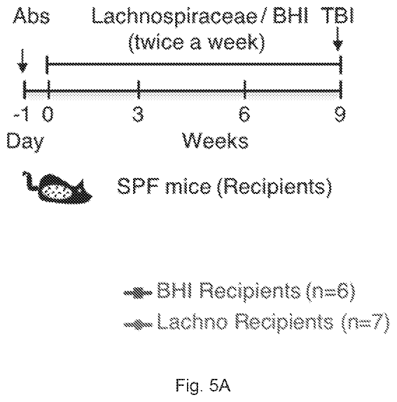

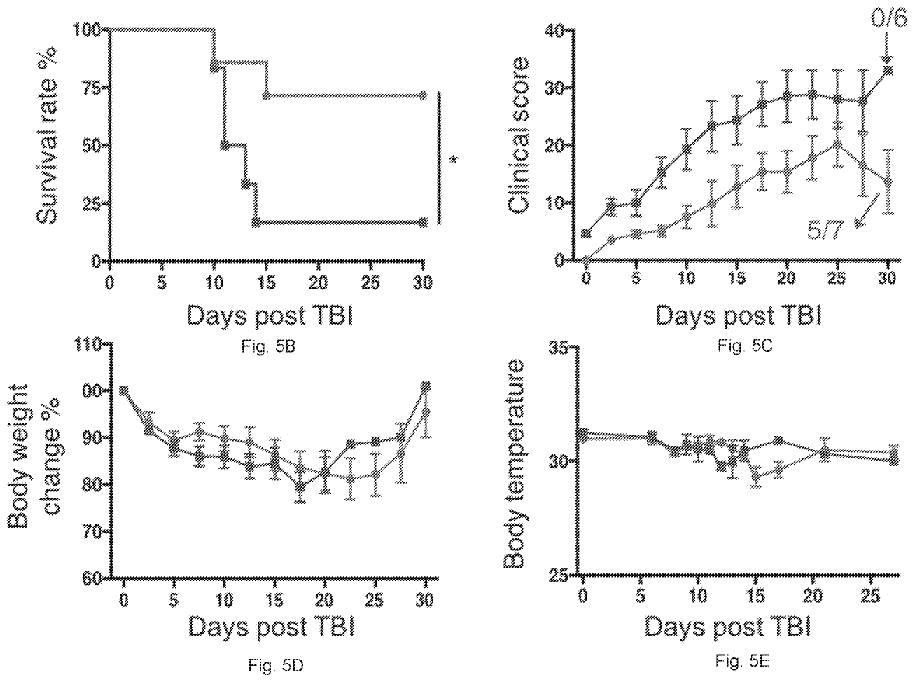

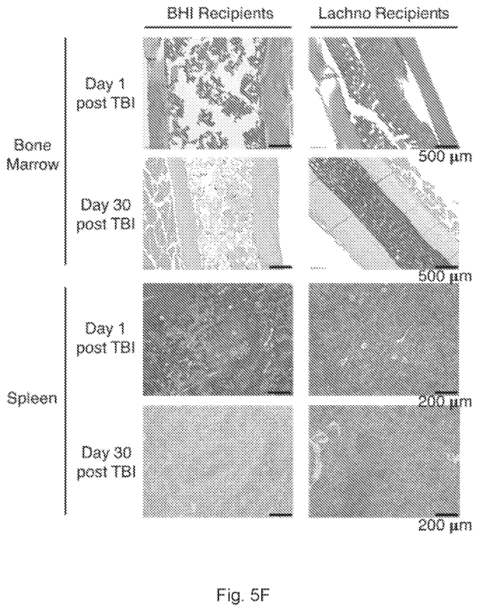

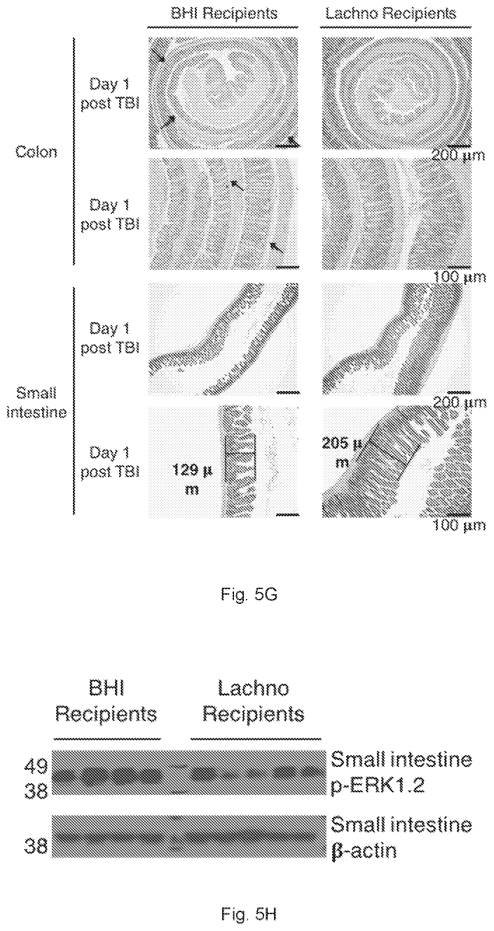

To characterize the nature of Lachnospiraceae in radiation process, SPF C57BL/6 mice were inoculated with a mixture of 23 Lachnospiraceae strains (Lachno) by oral gavage twice a week for 9 weeks (FIG. 5A). As controls, SPF C57BL/6 mice received the brain heart infusion (BHI) medium in which the bacteria were grown for the same procedure. Lachno recipients and BHI recipients both received lethal dose total body irradiation. The thirty-day survival of BHI recipients was 16.7% compared to 71.4% survival in Lachno recipients (FIG. 5B). Elevated survival in Lachno recipients was also associated with drastically decreased clinical score (FIG. 5C), while body weight and temperature showed no obvious difference between Lachno and BHI recipients (FIG. 5D-E). Histologic features of hematopoietic system were examined by haematoxylin and eosin (H&E) staining. As early as day 1 post TBI, there was more stromal injury and cell death in femurs and spleens from BHI recipients compared to that from Lachno recipients (FIG. 5F). At day 30 post TBI, appreciable atrophy and cell depletion were still observed in control recipients while femurs and spleens from Lachno recipients were practically normal in appearance. Next, gastrointestinal damage was assessed at day 1 post TBI. Colon sections from BHI recipients showed crypt distortion and atrophy, which was highlighted by gaps between crypt bases and muscularis mucosa, a common epithelial response to injury (FIG. 5G). However, all crypts attached closely to muscularis mucosae in Lachno recipients. Small intestine H&E staining revealed a dramatic shrinkage in intestinal villi from control recipients, which was greatly rescued by Lachnospiraceae administration. Additionally, Lachno recipients had reduced phosphorylation ERK in small intestines suggesting that Lachnospiraceae generated a less inflammatory environment in gastrointestinal system, which was in accord with less injury in this group (FIG. 5H). Furthermore, fluorescein isothiocyanate (FITC)-Dextran was used to examine whether Lachnospiraceae affected gut permeability in vivo and found that Lachno recipients showed reduced gut permeability compared to BHI recipients post TBI (FIG. 5I). Taken together, these results show that administration of Lachnospiraceae effectively attenuated radiation-induced hematopoietic and gastrointestinal syndrome.

### Example 6

### Commensal-Associated Short Chain Fatty Acid, Butyrate, Partially Ameliorated Acute Radiation Syndrome

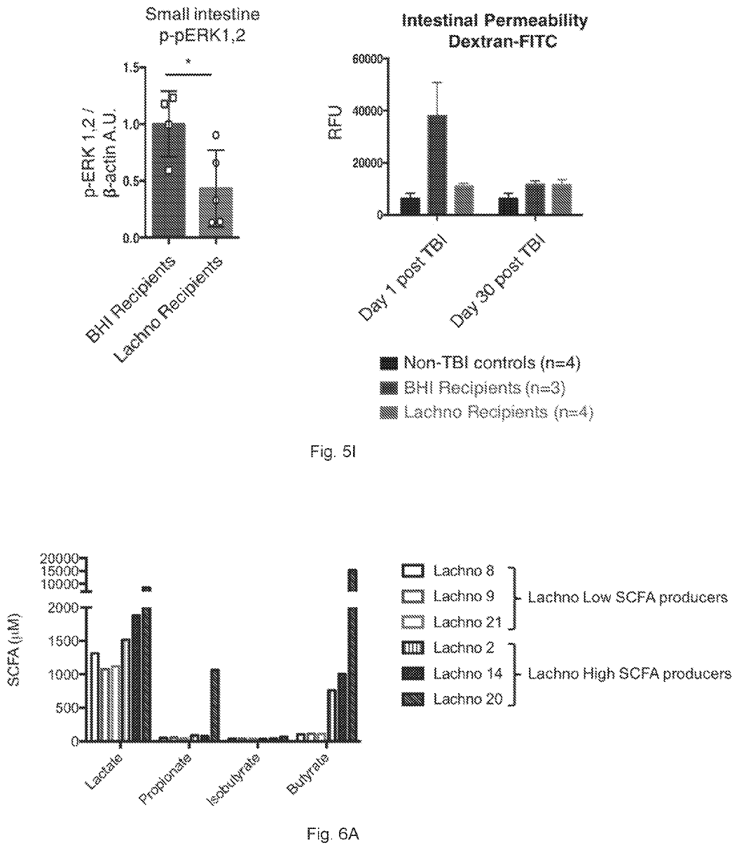

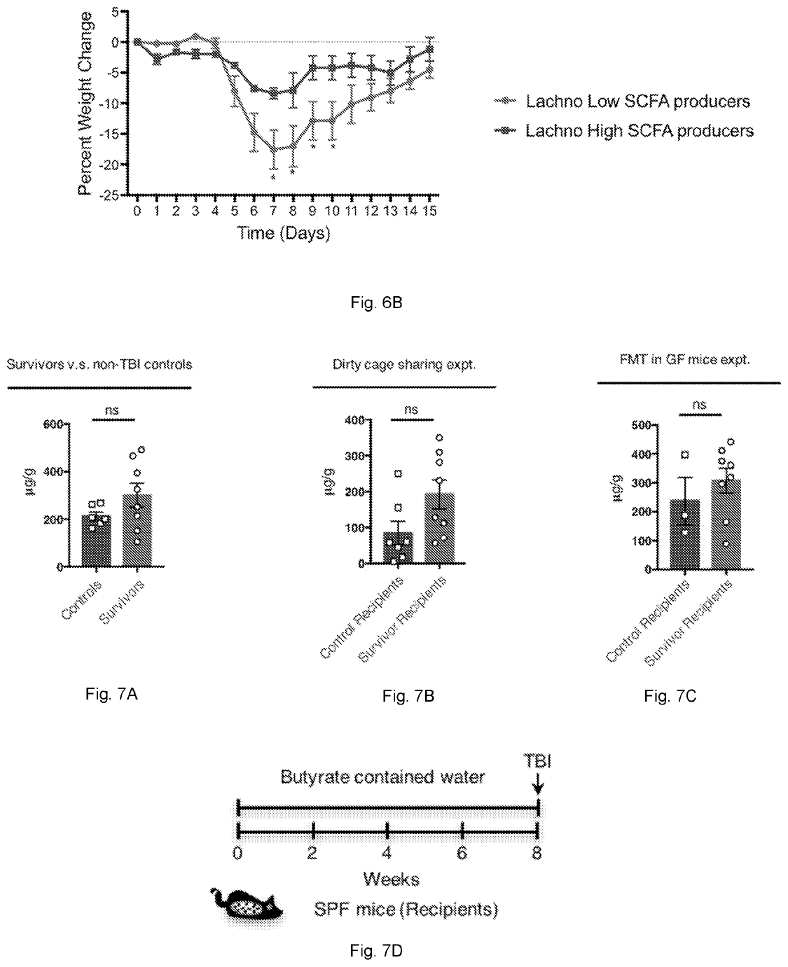

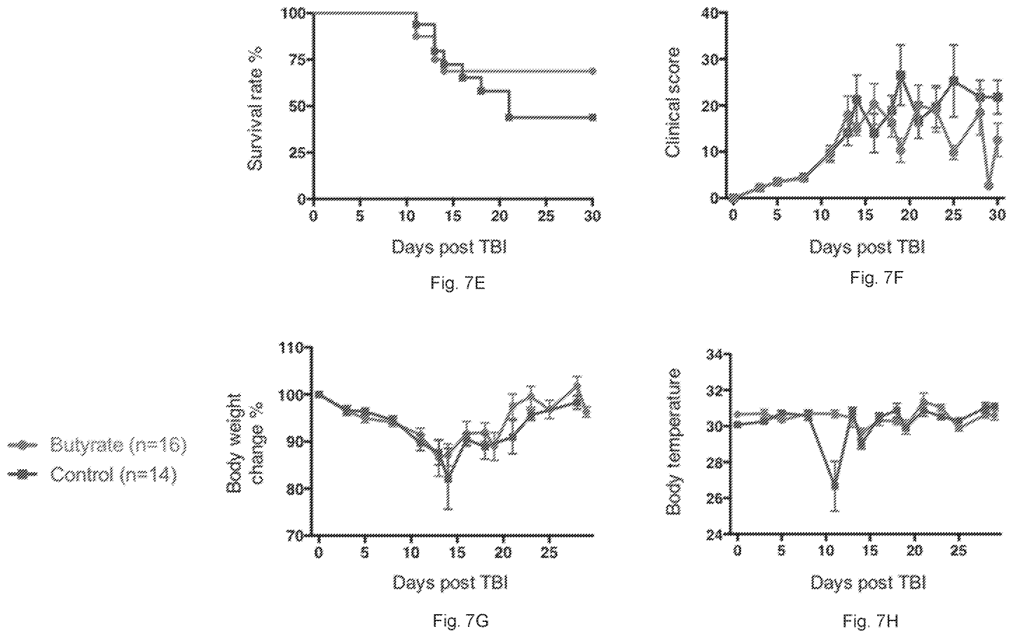

It is well established that Clostridiales and Lachnospiraceae bacterial groups produce short chain fatty acids (SCFAs) via fermentation of dietary polysaccharides³²⁻³⁴. SCFAs especially butyrate, which is the most commonly studied SCFA, are important substrates for maintaining intestinal epithelium and play a role in regulating immune system and inflammatory response. Increased abundance of Lachnospiraceae is expected to enhance the capability to produce SCFAs. To validate this hypothesis, the concentrations of lactate, propionate, isobutyrate and butyrate were detected in each individual Lachnospiraceae strain within the disclosed 23 stains pool. Here, for illustration and not intended to be limiting, six representative strains with three SCFAs high producers and three SCFAs low producers (FIG. 6A) are shown. These high producer strains, especially strain 20, exhibited a remarkable ability to produce butyrate and propionate. Lachnospiraceae strains, which produce butyrate higher than 120 μM and propionate higher than 60 μM, are expected to have better outcome in protecting against radiation-induced damage. It has previously been shown that Lachno with high, but not low levels, of SCFAs-production mitigated weight loss in DSS-induced colitis model (FIG. 6B). Butyrate concentrations in long-lived TBI survivors or survivor recipients were slightly but not significantly higher than that in non-TBI controls or control recipients (FIGS. 7A-C). To more precisely demonstrate butyrate's function, SPF C57BL/6 mice were treated with butyrate contained water for 8 weeks followed by total body irradiation (FIG. 7D). The thirty-day survival rate of butyrate recipients was 68% compared to 43% in control recipients (FIG. 7E) together with slightly lower clinical scores as well as body weight and temperature changes (FIGS. 7F-H). These results suggested that butyrate contributed to radio-resistance conducted by gut microbiota.

### Example 7

### Lachnospiraceae Improves or does not Mitigate the Therapeutic Efficacy of Irradiation in Tumor Models

Radiotherapy, using high dose ionizing radiation, is one of the most successful and widely used non-surgical therapies for the treatment of localized solid cancers³⁵. The success of radiotherapy in eradicating a tumor depends principally on the total radiation dose given. But high dose radiation will cause severe damage to normal tissues^{36,37} So, the key challenge in radiotherapy is to maximize radiation doses to cancer cells while decreasing side effects.

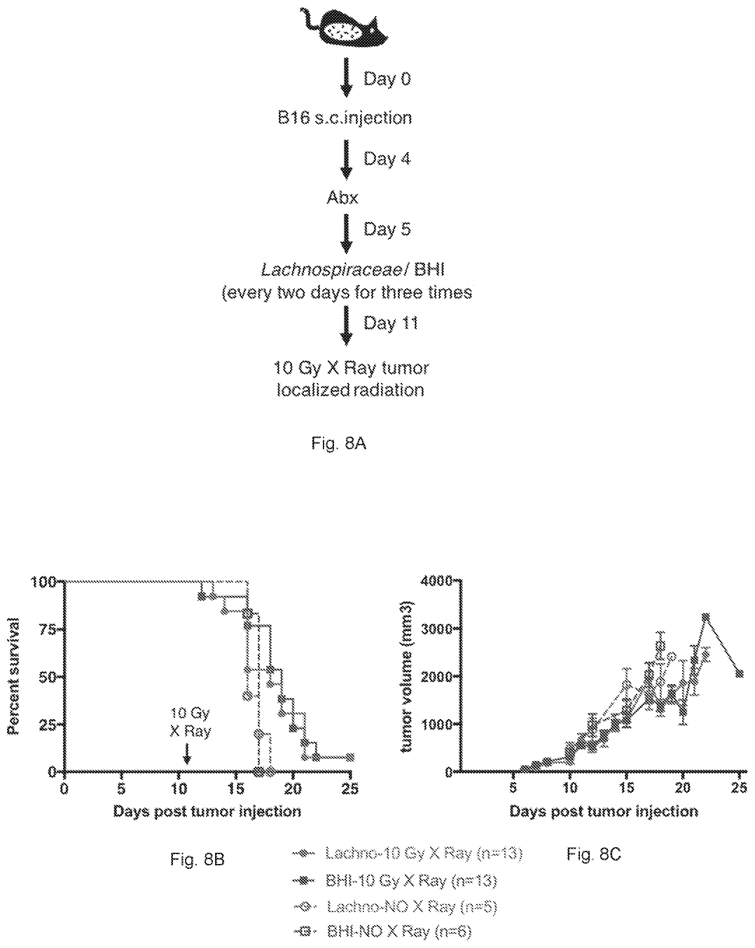

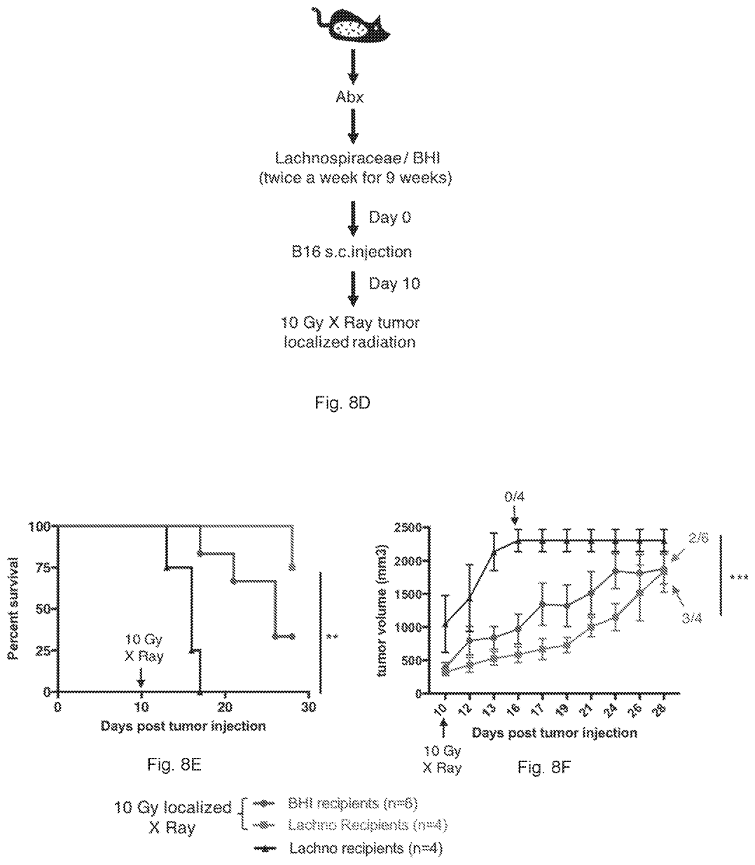

As the data herein showed a dramatic attenuation of radiation-induced damage by gut microbiota administration, efforts were undertaken to then investigate if microbiota and radiation combined therapy could successfully control tumor progress or at least does not affect the efficacy of radiotherapy. To this end, two strategies were employed, namely treating mice with Lachnospiraceae before or after tumor injection. As shown in FIG. 8A, SPF C57BL/6 mice were subcutaneously injected with B16 cells, a murine melanoma tumor cell line. Then, tumor-bearing mice were treated with Lachnospiraceae alone, BHI medium alone, Lachnospiraceae for 10 days followed by 10Gy X Ray localized radiation or BHI medium for 10 days followed by 10Gy X Ray localized radiation (FIG. 8A). Tumor volumes were measured.

Radiation in tumor-bearing mice caused longer survival both in Lachnospiraceae and BHI treated groups. But there was no difference in survival rate nor tumor volume between Lachn-10 Gy X Ray group and BHI-10 Gy X Ray group, which indicated that Lachnospiraceae did not negatively affect radiation efficacy (FIGS. 8B and 8C).

Because the B16 tumors were aggressive and grew very fast, there was a limited time interval for Lachnospiraceae transplantation. There was a concern that in this strategy, Lachnospiraceae did not have sufficient time to re-colonize the intestine. To overcome this problem, mice were treated with Lachnospiraceae before tumor injection for a longer period so that this bacterium could better colonize the intestine. As shown in FIG. 8D, SPF C57BL/6 mice were treated with Lachnospiraceae strains by oral gavage twice a week for 9 weeks. BHI medium was used as a control. B16 cells were then subcutaneously injected into Lachno recipients or BHI recipients, respectively. Mice were monitored until most of the tumors grew around 10 mm×10 mm in two dimensions and then given 10 Gy X Ray irradiation locally. Almost all of the Lachno recipients survived radiation, while all of the non-irradiated Lachno recipients died within 2-3 weeks of inoculation (FIGS. 8E and 8F). When Lachno and BHI treated groups that received radiation were compared, Lachno recipients exhibited a trend of reduced tumor growth with slower tumor volume increase as well as increased survivor rate post tumor inoculation (FIG. 8E, F). This suggests that sufficient microbiota transplant might be employed as a radio-protector to improve the outcome in cancer radiotherapy. These results demonstrate that depending on the condition of treatment, Lachnospiraceae either does not mitigate the efficacy of radiotherapy or improves radiotherapy efficacy and prohibits progression of an aggressive tumor model.

### Example 8

### Screening for Bacterial Strains that Produce High Levels of SCFAs

In some embodiments, disclosed herein are methods of screening strains to identify those that produce high levels of SCFAs. Such screening methods and systems can be useful in identifying strains that have similar mitigating and/or additive therapeutic effects as the exemplary strains disclosed herein.

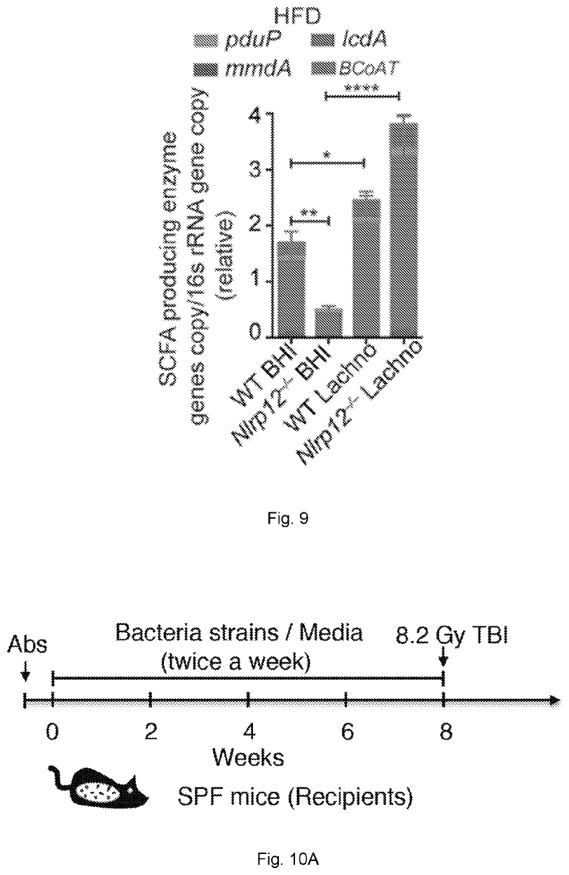

Clostridiales and Lachnospiraceae bacterial groups produce SCFAs via fermentation of dietary polysaccharides (Atarashi et al., 2013; den Besten et al., 2013; Reichardt et al., 2014). Increased abundance of Lachnospiraceae is expected to enhance the capability to produce SCFAs. The Lachnospiraceae mixture produced the SCFAs butyrate and propionate, but not isobutyrate, compared to the BHI medium. Dietary hexose and fucose can be used to generate the SCFA propionate by three independent pathways: succinate, acrylate, and propanediol. Key enzymes from bacteria that are important in these pathways include mmdA, encoding methylmalonyl-CoA decarboxylase for the succinate pathway; lcdA, encoding lactoyl-CoA dehydratase for the acrylate pathway; and pduP, encoding propionaldehyde dehydrogenase for the propanediol pathway. Additionally, BCoAT, encoding butyryl-CoA transferase, is essential for butyrate biosynthesis. Reduced expression of these enzymes correlates with reduced propionate and butyrate (Reichardt et al., 2014). The colonic microbiota from Nlrp12⁻⁻ on HFD showed significantly reduced copy numbers of these genes compared to similarly treated WT mice, while Lachnospiraceae treatment significantly increased these genes (FIG. 9). Since Lachnospiraceae produced SCFAs and also mitigated obesity in Nlrp12⁻⁻ mice, SCFAs were assessed to see if they could limit HFD-induced obesity in the Nlrp12⁻⁻ mice. Propionate and butyrate were given to WT and Nlrp12⁻⁻ mice on LFD or HFD via their drinking water ad libitum.

Thee data illustrate methods of screening strains producing relatively high levels of SCFA, and/or for markers of SCFA synthesis. Such screening methods and systems can comprise a composite analysis of the enzymes required for SCFA synthesis (FIG. 9). It was verified that the mouse strain which lacks lachno (bar that says Nlrp12⁻⁻ BHI—BHI is the blank media) has lower gene copy for SCFA producing enzymes. Conversely when these mice were fed with lachno, the gene copy for these enzymes went up (bar that says Nlrp12⁻⁻ lachno).

### Example 9

### SCFA High Producer Versus Low Producer-TBI Model

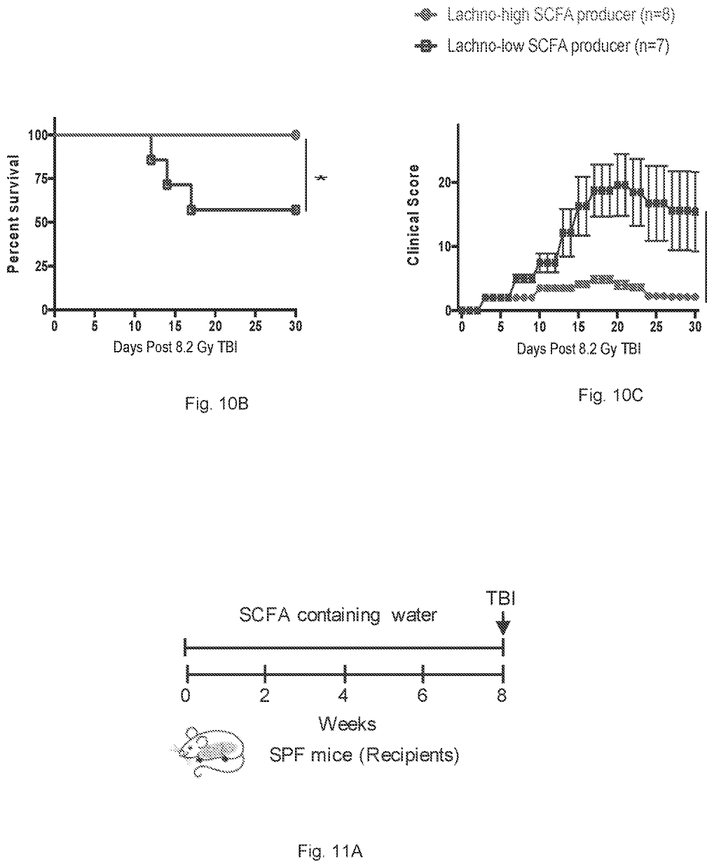

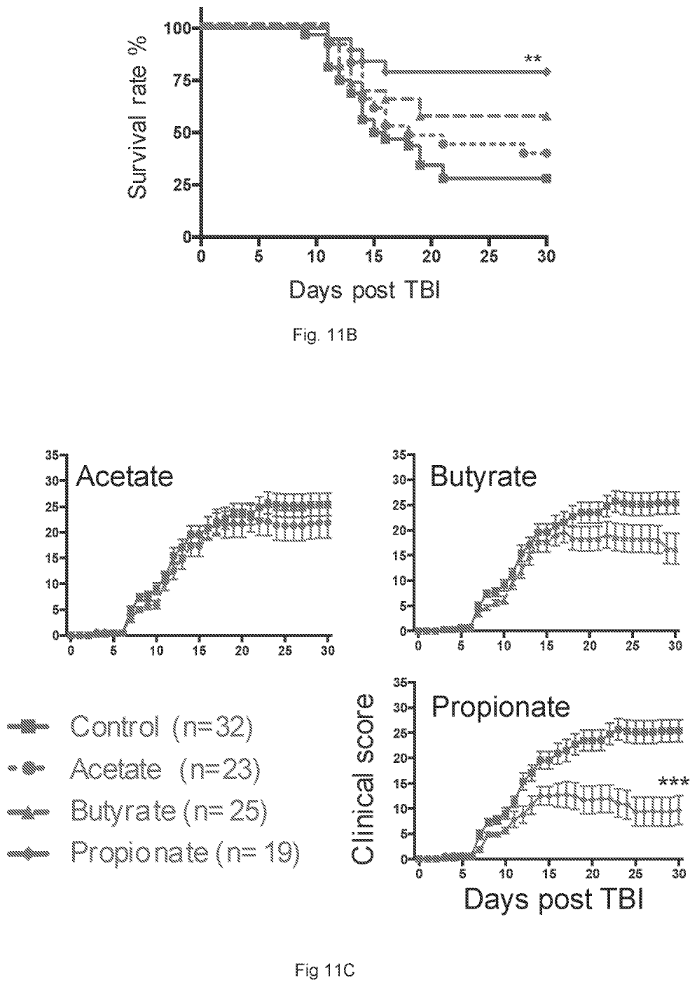

SCFA production was detected within 23 Lachnospiraceae strains, including 3 strains that were determined to produce high levels of SCFAs and 3 strains that produced low levels of SCFAs (FIGS. 10A-10C). These Lachno-high SCFA producer strains and low producer strains were transferred into SPF mice separately followed by lethal dose TBI. The survival rate and clinical scores showed that high-producer strains had a significant better protection against radiation, which further support the conclusion herein that SCFAs play an important role in radio-sensitivity.

### Example 10

### Three SCFAs Function in TBI Model (Propionate Shows Best Protection)

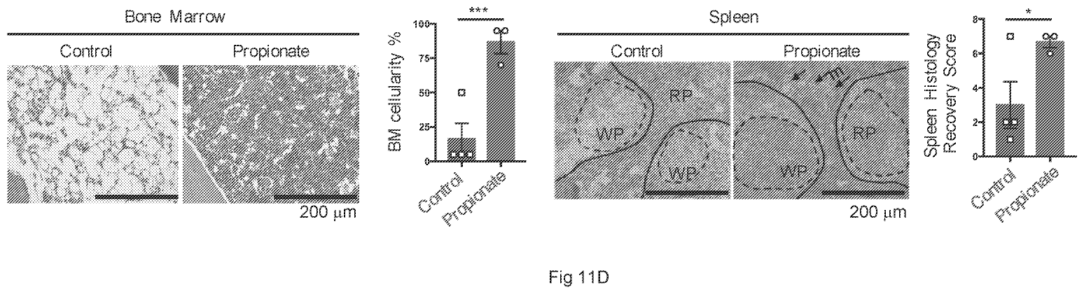

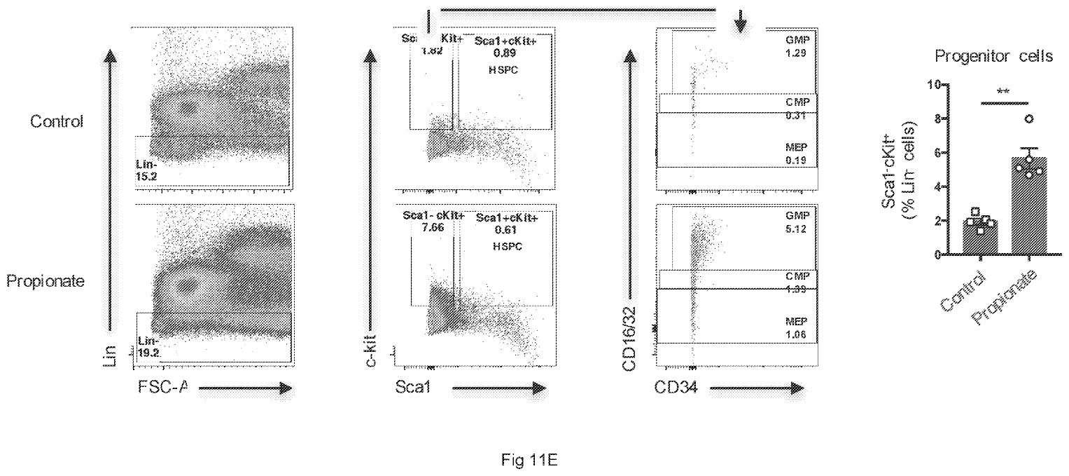

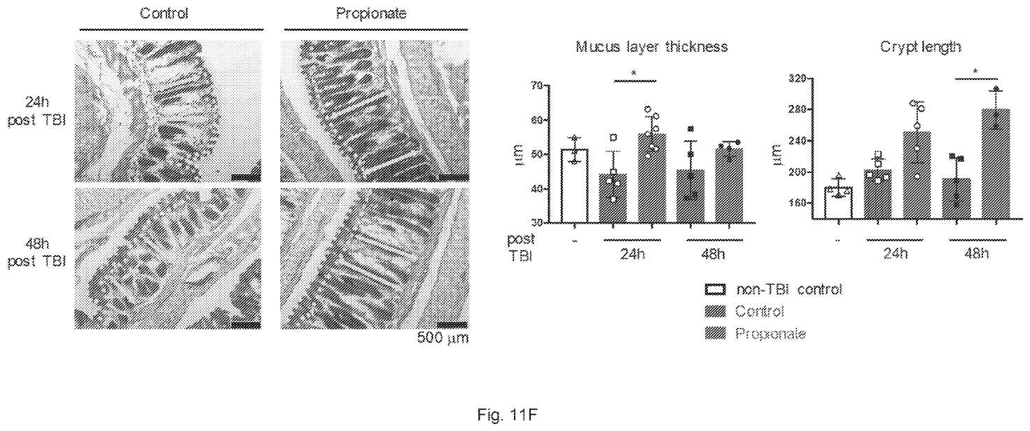

SPF C57BL/6 mice were treated with acetate, butyrate or propionate supplemented water for 8 weeks respectively, followed by a lethal dose TBI (FIG. 11A). Thirty-day survival rates of SCFA recipients were 79% in propionate-treated group compared to 28% in control group (FIG. 11B) accompanied by lower clinical scores (FIG. 11C). While, acetate and butyrate showed slight protection. Elevated bone marrow cellularity and splenic white and red pulp recovery were also observed in the propionate-treated group (FIG. 11D). Propionate treatment attenuated radiation-induced loss of granulocyte-macrophage progenitors (GMP), common myeloid progenitors (CMP) and megakaryocyte-erythroid progenitors (MEP), reflected as a significant increase in total Sca1⁻cKit⁺ progenitor cells compared to that of control recipients (FIG. 11E). To examine the effect of propionate on the gastrointestinal system, Alcian blue and periodic acid-Schiff (AB/PAS) staining of all intracellular mucin glycoproteins within goblet cells was completed. Results revealed significantly increased mucus thickness and crypt length in propionate recipients compared with control ones (FIG. 11F). These findings indicate that propionate leads to protection from hematopoietic and gastrointestinal syndromes.

### Example 11

### Different Combinations of SCFAs in TBI Model

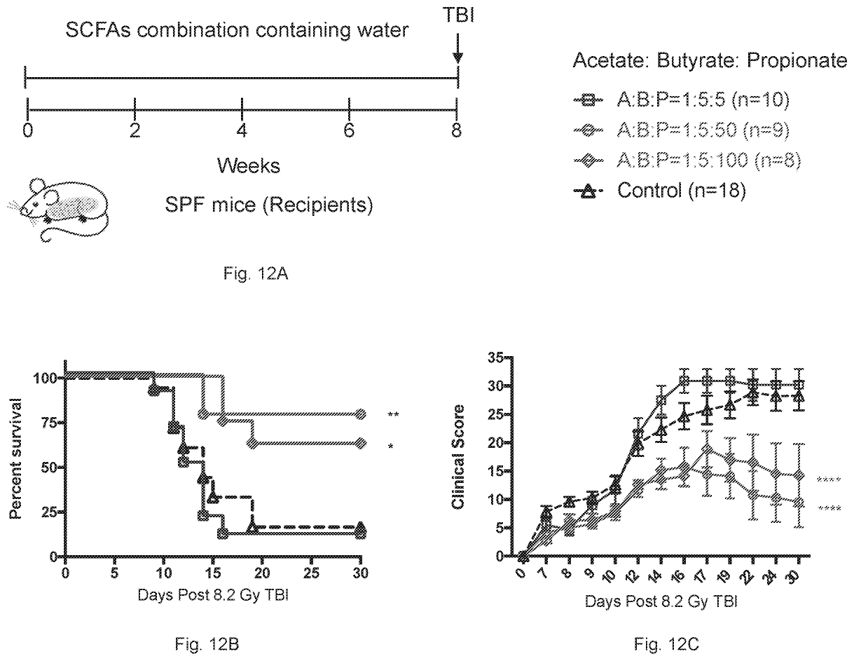

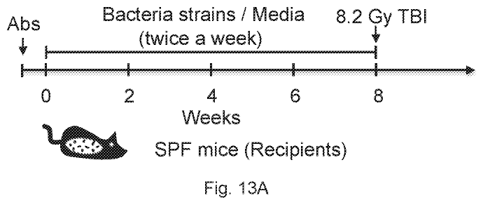

Acetate, butyrate and propionate were mixed by three different ratios and used to treat SPF C57BL/6 mice with these combinations for 8 weeks respectively, followed by a lethal dose TBI (FIG. 12A). Thirty-day survival rates were 78% and 63% in A:B:P=1:5:50 and 1:5:100 group compared to 17% in control group (FIG. 12B) accompanied by lower clinical scores (FIG. 12C).

### Example 12

### A New Bacteria Enterococcus can Also Protect Against Radiation-Induced Syndrome

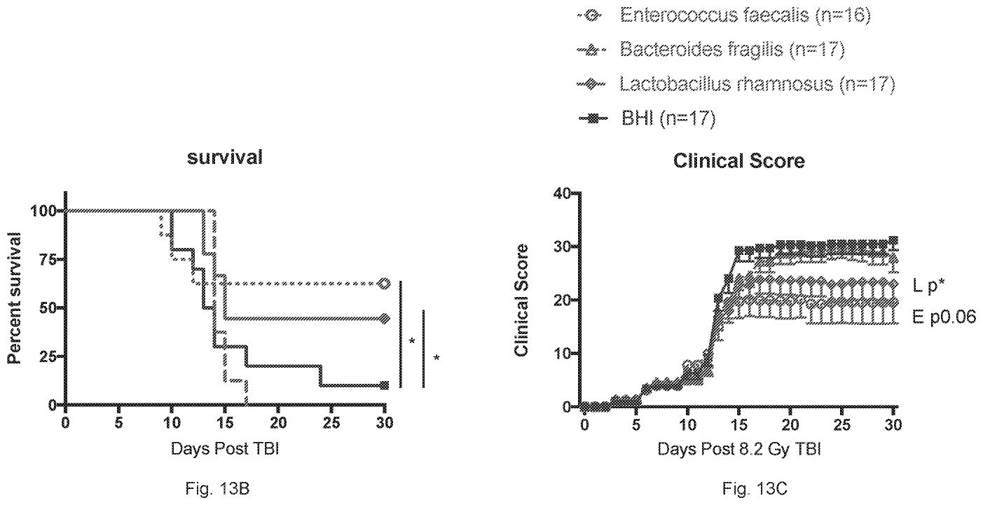

Two other bacteria strains (Enterococcus faecalis and Bacteroides fragilis) were tested, which were increased in elite-survivors detect by 16s rRNA sequencing, together with the well-known probiotics, Lactobacillus rhamonosus. These strains were cultured in vitro and separately transferred into SPF mice for 8 weeks, followed with lethal dose TBI and monitoring of the survival rate and clinical scores (FIG. 13A). The data shows Enterococcus faecalis and Lactobacillus rhamonosus both have a radioprotective function with a survival rate around 40%-60%, but not as dramatic as Lachnospiraceae (75% survival). See FIG. 13B-13C.

### Example 13

### Tryptophan Metabolites were Found as Novel Radio-Protectants by Untargeted Metabolomics Detection

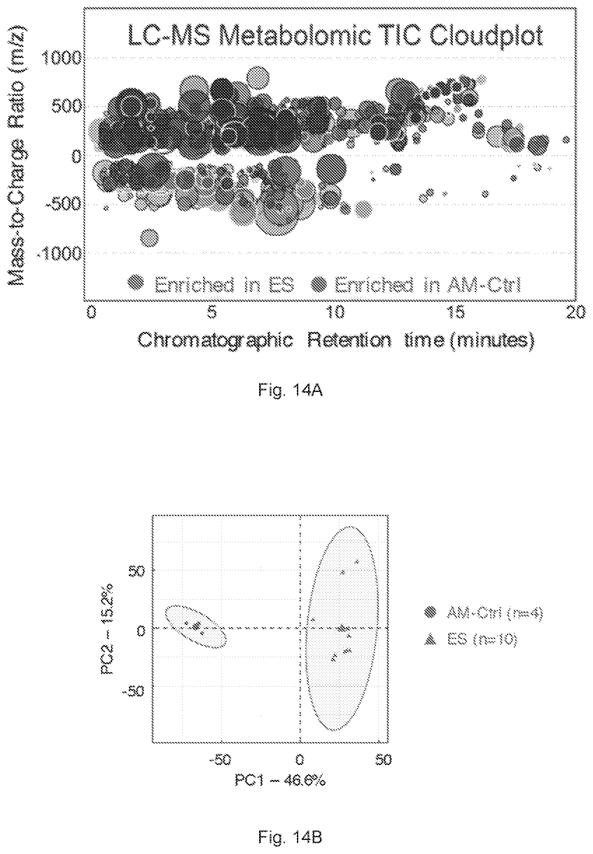

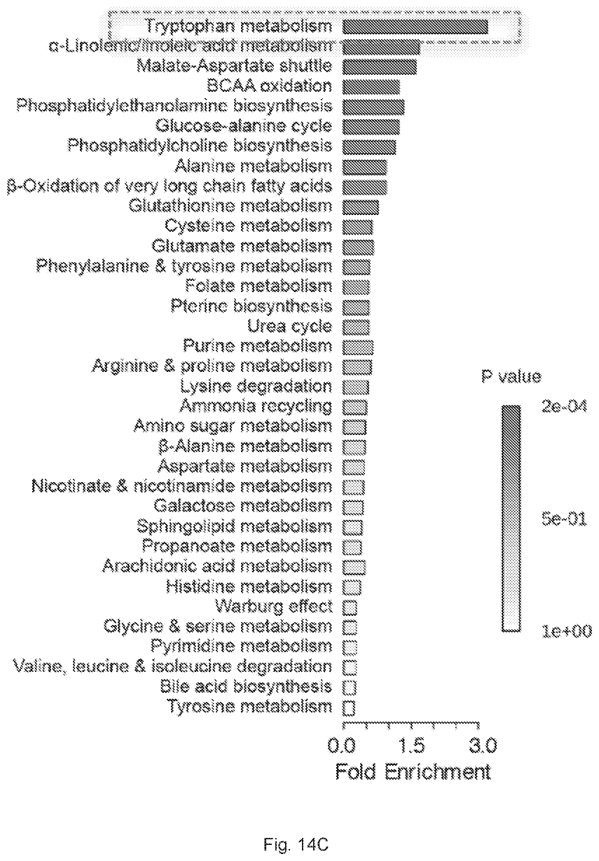

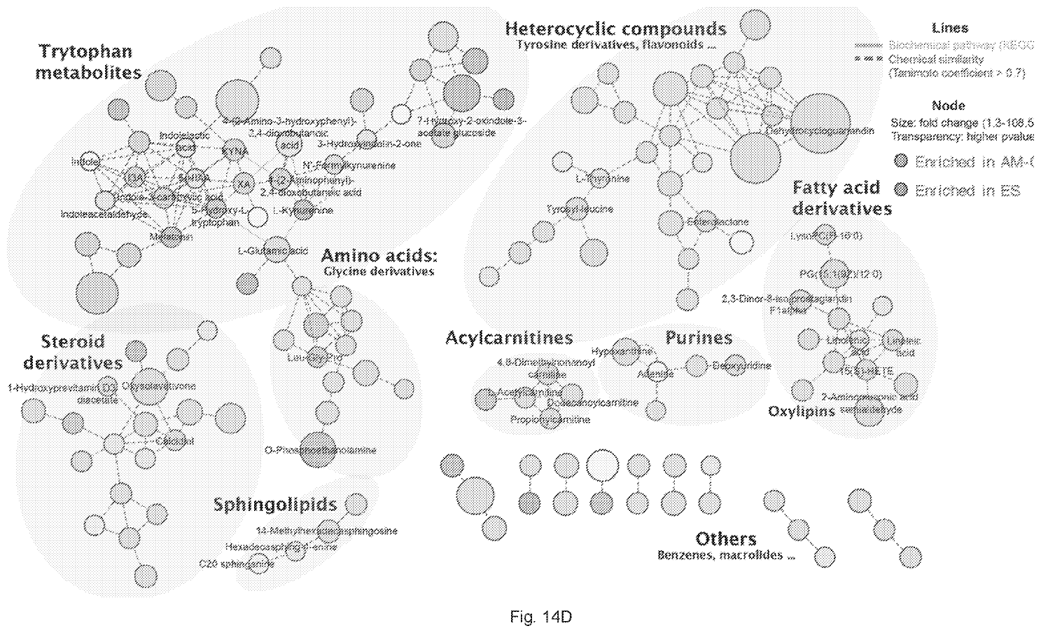

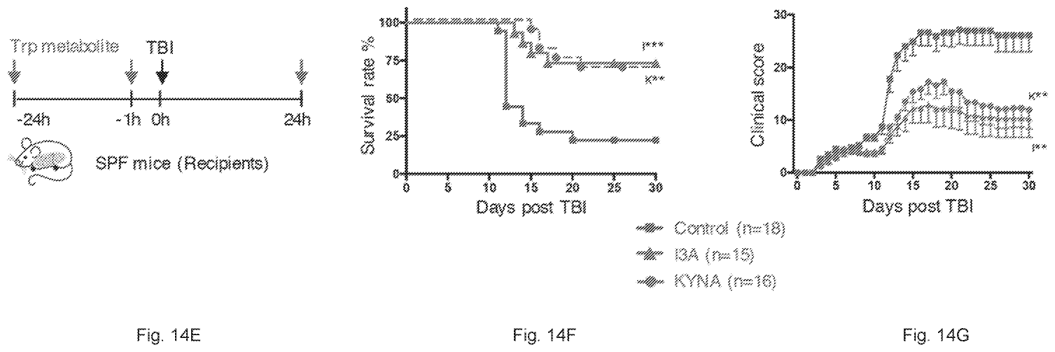

Besides propionate, a metabolomics approach was used to identify other metabolites with potentially protective or pathogenic consequences in an unbiased fashion (38, 39). An untargeted metabolomics of fecal samples from elite-survivors and AM-Ctrl on a high-resolution accurate mass (HRAM) mass spectrometry-based platform was performed (40). A total of 3787 ion features were detected as significantly altered (p<0.05, fold change>1.2) between elite-survivors and AM-Ctrl. Ion features of top 500 largest fold changes or of microbial relevance were fed into the chemoinformatic pipeline, resulting in 141 unique structures identified, including amino acids, fatty acids, steroid derivatives, acylcarnitines, saccharides, glycolytic and tricarboxylic acid cycle intermediates, and products of microbial metabolism, etc. Total ion chromatogram (TIC) metabolomic cloudplot and principal component analysis (PCA) score plot showed that the metabolite profiles were dramatically distinct between these two groups (FIG. 14A-B). Compared with AM-Ctrl samples, the most highly enriched metabolites from elite-survivor feces clustered in the tryptophan (Trp) metabolic pathway with 5- to 8-fold changes in indole-3-carboxaldehyde (I3A) and kynurenic acid (KYNA) (FIG. 14C-D). The function of these Trp metabolites in radiomitigation in vivo was investigated. Both metabolites led to significant enhanced survivals in SPF mice, which received Trp metabolites and lethal radiation (FIG. 14E-G). The I3A and KYNA treated groups both had survival rates of around 75%, indicating Trp metabolites were also potent in attenuating radiation-induced damage.

### IV. REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1 Waselenko, J. K. et al. Medical management of the acute radiation syndrome: recommendations of the Strategic National Stockpile Radiation Working Group. Ann Intern Med 140, 1037-1051 (2004).
2 Dorr, H. & Meineke, V. Acute radiation syndrome caused by accidental radiation exposure—therapeutic principles. BMC Med 9, 126, doi:10.1186/1741-7015-9-126 (2011).
3 Ghosh, S. P. et al. Amelioration of radiation-induced hematopoietic and gastrointestinal damage by Ex-RAD(R) in mice. J Radiat Res 53, 526-536, doi:10.1093/jrr/rrs001 (2012).
4 Shadad, A. K., Sullivan, F. J., Martin, J. D. & Egan, L. J. Gastrointestinal radiation injury: prevention and treatment. World J Gastroenterol 19, 199-208, doi:10.3748/wjg.v19.i2.199 (2013).
5 Mauch, P. et al. Hematopoietic stem cell compartment: acute and late effects of radiation therapy and chemotherapy. Int J Radiat Oncol Biol Phys 31, 1319-1339, doi:10.1016/0360-3016(94)00430-5 (1995).
6 Singh, V. K., Newman, V. L. & Seed, T. M. Colony-stimulating factors for the treatment of the hematopoietic component of the acute radiation syndrome (H-ARS): a review. Cytokine 71, 22-37, doi:10.1016/j.cyto.2014.08.003 (2015).
7 Schwartz, G. N., Vigneulle, R. M. & MacVittie, T. J. Survival of erythroid burst-forming units and erythroid colony-forming units in canine bone marrow cells exposed in vitro to 1 MeV neutron radiation or X rays. Radiat Res 108, 336-347 (1986).
8 Imai, Y. & Nakao, I. In vivo radiosensitivity and recovery pattern of the hematopoietic precursor cells and stem cells in mouse bone marrow. Exp Hematol 15, 890-895 (1987).
9 Coleman, C. N., Stone, H. B., Moulder, J. E. & Pellmar, T. C. Medicine. Modulation of radiation injury. Science 304, 693-694, doi:10.1126/science.1095956 (2004).
10 Geiger, H. et al. Pharmacological targeting of the thrombomodulin-activated protein C pathway mitigates radiation toxicity. Nat Med 18, 1123-1129, doi:10.1038/nm.2813 (2012).
11 Gudkov, A. V. & Komarova, E. A. The role of p53 in determining sensitivity to radiotherapy. Nat Rev Cancer 3, 117-129, doi:10.1038/nrc992 (2003).
12 Singh, V. K., Romaine, P. L. & Seed, T. M. Medical Countermeasures for Radiation Exposure and Related Injuries: Characterization of Medicines, FDA-Approval Status and Inclusion into the Strategic National Stockpile. Health Phys 108, 607-630, doi:10.1097/HP.0000000000000279 (2015).
13 Kau, A. L., Ahern, P. P., Griffin, N. W., Goodman, A. L. & Gordon, J. I. Human nutrition, the gut microbiome and the immune system. Nature 474, 327-336, doi:10.1038/nature10213 (2011).
14 Ridaura, V. K. et al. Gut microbiota from twins discordant for obesity modulate metabolism in mice. Science 341, 1241214, doi:10.1126/science.1241214 (2013).
15 Aron-Wisnewsky, J. & Clement, K. The gut microbiome, diet, and links to cardiometabolic and chronic disorders. Nat Rev Nephrol 12, 169-181, doi:10.1038/nrneph.2015.191 (2016).
16 Dickson, R. P. The microbiome and critical illness. Lancet Respir Med 4, 59-72, doi:10.1016/S2213-2600(15)00427-0 (2016).
17 Schaubeck, M. et al. Dysbiotic gut microbiota causes transmissible Crohn's disease-like ileitis independent of failure in antimicrobial defence. Gut 65, 225-237, doi:10.1136/gutjnl-2015-309333 (2016).
18 Chen, L. et al. NLRP12 attenuates colon inflammation by maintaining colonic microbial diversity and promoting protective commensal bacterial growth. Nat Immunol 18, 541-551, doi:10.1038/ni.3690 (2017).
19 Ciorba, M. A. & Stenson, W. F. Probiotic therapy in radiation-induced intestinal injury and repair. Ann N Y Acad Sci 1165, 190-194, doi:10.1111/j.1749-6632.2009.04029.x (2009).
20 Ciorba, M. A. et al. Lactobacillus probiotic protects intestinal epithelium from radiation injury in a TLR-2/cyclo-oxygenase-2-dependent manner. Gut 61, 829-838, doi:10.1136/gutjnl-2011-300367 (2012).
21 Crawford, P. A. & Gordon, J. I. Microbial regulation of intestinal radiosensitivity. Proc Natl Acad Sci USA 102, 13254-13259, doi:10.1073/pnas.0504830102 (2005).
22 Lam, V. et al. Intestinal microbiota as novel biomarkers of prior radiation exposure. Radiat Res 177, 573-583 (2012).
23 van Vliet, M. J., Harmsen, H. J., de Bont, E. S. & Tissing, W. J. The role of intestinal microbiota in the development and severity of chemotherapy-induced mucositis. PLoS Pathog 6, e1000879, doi:10.1371/journal.ppat.1000879 (2010).
24 Wang, A. et al. Gut microbial dysbiosis may predict diarrhea and fatigue in patients undergoing pelvic cancer radiotherapy: a pilot study. PLoS One 10, e0126312, doi:10.1371/journal.pone.0126312 (2015).
25 Manichanh, C. et al. The gut microbiota predispose to the pathophysiology of acute postradiotherapy diarrhea. Am J Gastroenterol 103, 1754-1761, doi:10.1111/j.1572-0241.2008.01868.x (2008).
26 Kurkjian, C. J. et al. The Toll-Like Receptor 2/6 Agonist, FSL-1 Lipopeptide, Therapeutically Mitigates Acute Radiation Syndrome. Sci Rep 7, 17355, doi:10.1038/s41598-017-17729-9 (2017).
27 Carmody, R. N. et al. Diet dominates host genotype in shaping the murine gut microbiota. Cell Host Microbe 17, 72-84, doi:10.1016/j.chom.2014.11.010 (2015).
28 Smith, C. C., Snowberg, L. K., Gregory Caporaso, J., Knight, R. & Bolnick, D. I. Dietary input of microbes and host genetic variation shape among-population differences in stickleback gut microbiota. ISME J 9, 2515-2526, doi:10.1038/ismej.2015.64 (2015).
29 Mettler, F. A., Jr. & Voelz, G. L. Major radiation exposure—what to expect and how to respond. N Engl J Med 346, 1554-1561, doi:10.1056/NEJMra000365 (2002).
30 Reeves, A. E., Koenigsknecht, M. J., Bergin, I. L. & Young, V. B. Suppression of Clostridium difficile in the gastrointestinal tracts of germfree mice inoculated with a murine isolate from the family Lachnospiraceae. Infect Immun 80, 3786-3794, doi:10.1128/IAI.00647-12 (2012).
31 Nakanishi, Y., Sato, T. & Ohteki, T. Commensal Gram-positive bacteria initiates colitis by inducing monocyte/macrophage mobilization. Mucosal Immunol 8, 152-160, doi: 10.1038/mi.2014.53 (2015).
32 Atarashi, K. et al. Treg induction by a rationally selected mixture of Clostridia strains from the human microbiota. Nature 500, 232-236, doi:10.1038/nature12331 (2013).
33 Reichardt, N. et al. Phylogenetic distribution of three pathways for propionate production within the human gut microbiota. ISME J 8, 1323-1335, doi:10.1038/ismej.2014.14 (2014).
34 den Besten, G. et al. The role of short-chain fatty acids in the interplay between diet, gut microbiota, and host energy metabolism. J Lipid Res 54, 2325-2340, doi:10.1194/jlr.R036012 (2013).
35 Schaue, D. & McBride, W. H. Opportunities and challenges of radiotherapy for treating cancer. Nat Rev Clin Oncol 12, 527-540, doi:10.1038/nrclinonc.2015.120 (2015).
36 Barnett, G. C. et al. Normal tissue reactions to radiotherapy: towards tailoring treatment dose by genotype. Nat Rev Cancer 9, 134-142, doi:10.1038/nrc2587 (2009).
37 Formenti, S. C. & Demaria, S. Systemic effects of local radiotherapy. Lancet Oncol 10, 718-726, doi:10.1016/S1470-2045(09)70082-8 (2009).
38 M. G. Rooks, W. S. Garrett, Gut microbiota, metabolites and host immunity. Nat Rev Immunol 16, 341-352 (2016).
39 M. Levy, E. Blacher, E. Elinav, Microbiome, metabolites and host immunity. Curr Opin Microbiol 35, 8-15 (2017).
40 Z. Lai et al., Identifying metabolites by integrating metabolome databases with mass spectrometry cheminformatics. Nat Methods 15, 53-56 (2018).

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of mitigating and/or preventing side effects from radiation therapy, the method comprising:
providing a subject to be treated with radiation therapy, and/or a subject already treated with radiation therapy; and
administering to the subject a bacterium and/or metabolite thereof, wherein the bacterium comprises one or more bacterial strains capable of producing short chain fatty acids (SCFAs),
wherein side effects from radiation therapy are mitigated and/or prevented in the subject.

2. The method of claim 1, wherein the bacterium comprises intestinal microbiota.

3. The method of claim 1, wherein the SCFAs produced by the bacterial strains comprise acetate, butyrate and propionate, optionally wherein the ratio of acetate to butyrate to propionate is about 1:5:50, optionally about 1:5:100.

4. The method of any of claims 1 to 2, wherein the bacterium comprises strains selected from Lachnospiraceae, Enterococcus faecalis, Lactobacillus rhamonosusl, and combinations thereof.

5. The method of any of claims 1 to 4, wherein the bacterium comprises Lachnospiraceae strains, optionally wherein the Lachnospiraceae strains produce butyrate higher than about 120 μM and propionate higher than about 60 μM.

6. The method of any of claims 1 to 5, wherein the metabolite comprises one or more tryptophan metabolites.

7. The method of any of claims 1 to 6, wherein the subject is suffering from acute radiation syndrome (ARS), hematopoietic (HP) injury, gastrointestinal (GI) injury, cerebrovascular syndrome, cutaneous toxicity, pulmonary toxicity, cardiac toxicity and/or combinations thereof.

8. The method of any of claims 1 to 7, wherein administration of the bacterium and/or metabolite thereof effectively attenuates radiation-induced hematopoietic and/or gastrointestinal syndrome.

9. The method of any of claims 1 to 8, wherein the administration of the bacterium and/or metabolite to the subject occurs before or after radiation therapy.

10. The method of any of claims 1 to 9, wherein the bacterium and/or metabolite thereof is administered orally or by suppository.

11. The method of any of claims 1 to 10, wherein the subject is a human, optionally wherein the subject is suffering from a cancer, tumor or related condition.

12. A method of treating a tumor and/or a cancer in a subject, the method comprising:
administering radiation therapy to a subject in need; and
administering to the subject a bacterium and/or metabolite thereof, wherein the bacterium comprises one or more bacterial strains capable of producing SCFAs,
wherein the tumor and/or a cancer is treated, wherein the effectiveness of the treatment of the tumor and/or cancer is enhanced as compared to radiation therapy alone.

13. The method of claim 12, wherein the bacterium comprises intestinal microbiota.

14. The method of claim 12, wherein the SCFAs produced by the bacterial strains comprise acetate, butyrate and propionate, optionally wherein the ratio of acetate to butyrate to propionate is about 1:5:50, optionally about 1:5:100.

15. The method of any of claims 12 to 14, wherein the bacterium comprises strains selected from Lachnospiraceae, Enterococcus faecalis, Lactobacillus rhamonosusl, and combinations thereof.

16. The method of any of claims 12 to 15, wherein the bacterium comprises Lachnospiraceae strains, optionally wherein the Lachnospiraceae strains produce butyrate higher than about 120 μM and propionate higher than about 60 μM.

17. The method of claim 12, wherein the metabolite comprises one or more tryptophan metabolites.

18. The method of any of claims 12 to 17, wherein administration of the bacterium and/or metabolite thereof effectively attenuates radiation-induced hematopoietic and/or gastrointestinal syndrome.

19. The method of any of claims 12 to 18, wherein the administration of the bacterium and/or metabolite to the subject occurs before or after radiation therapy.

20. The method of any of claims 12 to 19, wherein the bacterium and/or metabolite thereof is administered orally or by suppository.

21. The method of any of claims 12 to 20, wherein the subject is a human, optionally wherein the subject is suffering from a cancer, tumor or related condition.

22. A method of treating and/or mitigating damage to a hematopoietic and/or gastrointestinal system in a subject, the method comprising administering to the subject a bacterium and/or metabolite thereof, wherein the bacterium comprises one or more bacterial strains capable of producing SCFAs.

23. The method of claim 22, wherein the administration of the bacterium and/or metabolite to the subject occurs before or after an event causing or potentially causing damage to the hematopoietic and/or gastrointestinal system of the subject.

24. The method of claim 22, wherein the event causing damage to the hematopoietic and/or gastrointestinal system includes radiation, chemotherapy and/or any event, therapy or exposure causing hematopoietic loss and/or acute radiation enteritis.

25. The method of any of claims 22 to 24, wherein administration of the bacterium and/or metabolite thereof effectively attenuates bone marrow loss due to exposure to radiation, chemotherapy or other therapy.

26. The method of any of claims 22 to 25, wherein the bacterium comprises intestinal microbiota.

27. The method of any of claims 22 to 26, wherein the SCFAs produced by the bacterial strains comprise acetate, butyrate and propionate, optionally wherein the ratio of acetate to butyrate to propionate is about 1:5:50, optionally about 1:5:100.

28. The method of any of claims 22 to 27, wherein the bacterium comprises strains selected from Lachnospiraceae, Enterococcus faecalis, Lactobacillus rhamonosusl, and combinations thereof.

29. The method of any of claims 22 to 28, wherein the bacterium comprises Lachnospiraceae strains, optionally wherein the Lachnospiraceae strains produce butyrate higher than about 120 μM and propionate higher than about 60 μM.

30. The method of any of claims 22 to 29, wherein the metabolite comprises one or more tryptophan metabolites.

31. An adjuvant therapeutic composition, the composition comprising:
a bacterium and/or metabolite thereof, wherein the bacterium comprises one or more bacterial strains capable of producing SCFAs; and
a therapeutically acceptable carrier.

32. The adjuvant therapeutic composition of claim 31, wherein the bacterium comprises intestinal microbiota.

33. The adjuvant therapeutic composition of claim 31, wherein the SCFAs produced by the bacterial strains comprise acetate, butyrate and propionate, optionally wherein the ratio of acetate to butyrate to propionate is about 1:5:50, optionally about 1:5:100.

34. The adjuvant therapeutic composition of any of claims 31 to 33, wherein the bacterium comprises strains selected from Lachnospiraceae, Enterococcus faecalis, Lactobacillus rhamonosusl, and combinations thereof.

35. The adjuvant therapeutic composition of any of claims 31 to 34, wherein the bacterium comprises Lachnospiraceae strains, optionally wherein the Lachnospiraceae strains produce butyrate higher than about 120 μM and propionate higher than about 60 μM.

36. The adjuvant therapeutic composition of any of claims 31 to 35, wherein the metabolite comprises one or more tryptophan metabolites.

37. The adjuvant therapeutic composition of any of claims 31 to 36, wherein the composition is configured as an adjuvant to anti-cancer radiation therapy and/or anti-cancer chemotherapy, optionally wherein the composition is configured to treat and/or mitigate damage to a hematopoietic and/or gastrointestinal system in a subject to which it is administered.

38. A method of screening bacterial strains for use as an anti-cancer adjuvant therapeutic, the method comprising:
providing one or more bacterial strains to be screened;
conducting a composite genomic analysis for enzymes required for SCFA synthesis; and
identify those bacterial strains with a relatively high gene copy for SCFA producing enzymes.

39. The method of claim 38, wherein the genes for SCFA producing enzymes comprise mmdA, encoding methylmalonyl-CoA decarboxylase for the succinate pathway; lcdA, encoding lactoyl-CoA dehydratase for the acrylate pathway; pduP, encoding propionaldehyde dehydrogenase for the propanediol pathway; and BCoAT, encoding butyryl-CoA transferase for butyrate biosynthesis.

40. The method of claim 38, wherein the one or more bacterial strains comprises intestinal microbiota.

41. The method of claim 38, wherein the SCFA producing enzymes produce SCFAs selected from acetate, butyrate and propionate.

42. The method of claim 38, wherein the bacterial strains are selected from Lachnospiraceae, Enterococcus faecalis, Lactobacillus rhamonosusl, and combinations thereof.
